SEQUENCE LISTING (32 sequences)

SEQ ID NO: 1 - protein, 9 aa, Artificial Sequence
  Description of Artificial Sequence Synthetic peptide
IISAVVGIL

SEQ ID NO: 2 - protein, 9 aa, Artificial Sequence
  Description of Artificial Sequence Synthetic peptide
KIFGSLAFL

SEQ ID NO: 3 - protein, 10 aa, Artificial Sequence
  Description of Artificial Sequence Synthetic peptide
YMLDLQPETT

SEQ ID NO: 4 - protein, 14 aa, Artificial Sequence
  Description of Artificial Sequence Synthetic peptide
PDTRPAPGSTAPPA

SEQ ID NO: 5 - protein, 6 aa, Artificial Sequence
  Description of Artificial Sequence Synthetic peptide
HGVTSA

SEQ ID NO: 6 - protein, 9 aa, Artificial Sequence
  Description of Artificial Sequence Synthetic peptide
EADPTGHSY

SEQ ID NO: 7 - protein, 9 aa, Artificial Sequence
  Description of Artificial Sequence Synthetic peptide
SAYGEPRKL

SEQ ID NO: 8 - protein, 9 aa, Artificial Sequence
  Description of Artificial Sequence Synthetic peptide
EVDPIGHLY

SEQ ID NO: 9 - protein, 9 aa, Artificial Sequence
  Description of Artificial Sequence Synthetic peptide
FLWGPRALV

SEQ ID NO: 10 - protein, 9 aa, Artificial Sequence
  Description of Artificial Sequence Synthetic peptide
AARAVFLAL

SEQ ID NO: 11 - protein, 8 aa, Artificial Sequence
  Description of Artificial Sequence Synthetic peptide
YRPRPRRY

SEQ ID NO: 12 - protein, 9 aa, Artificial Sequence
  Description of Artificial Sequence Synthetic peptide
VLPDVFIRC

SEQ ID NO: 13 - protein, 9 aa, Artificial Sequence
  Description of Artificial Sequence Synthetic peptide
AYGLDFYIL

SEQ ID NO: 14 - protein, 9 aa, Artificial Sequence
  Description of Artificial Sequence Synthetic peptide
KTWGQYWQV

SEQ ID NO: 15 - protein, 9 aa, Artificial Sequence
  Description of Artificial Sequence Synthetic peptide
ITDQVPFSV

SEQ ID NO: 16 - protein, 9 aa, Artificial Sequence
  Description of Artificial Sequence Synthetic peptide
YLEPGPVTA

SEQ ID NO: 17 - protein, 10 aa, Artificial Sequence
  Description of Artificial Sequence Synthetic peptide
LLDGTATLRL

SEQ ID NO: 18 - protein, 10 aa, Artificial Sequence
  Description of Artificial Sequence Synthetic peptide
VLYRYGSFSV

SEQ ID NO: 19 - protein, 9 aa, Artificial Sequence
  Description of Artificial Sequence Synthetic peptide
AAGIGILTV

SEQ ID NO: 20 - protein, 9 aa, Artificial Sequence
  Description of Artificial Sequence Synthetic peptide
ILTVILGVL

SEQ ID NO: 21 - protein, 9 aa, Artificial Sequence
  Description of Artificial Sequence Synthetic peptide
MSLQRQFLR

SEQ ID NO: 22 - protein, 9 aa, Artificial Sequence
  Description of Artificial Sequence Synthetic peptide
MLLAVLYCL

SEQ ID NO: 23 - protein, 9 aa, Artificial Sequence
  Description of Artificial Sequence Synthetic peptide
YMNGTMSQV

SEQ ID NO: 24 - protein, 9 aa, Artificial Sequence
  Description of Artificial Sequence Synthetic peptide
SEIWRDIDF

SEQ ID NO: 25 - protein, 9 aa, Artificial Sequence
  Description of Artificial Sequence Synthetic peptide
AFLPWHRLF

SEQ ID NO: 26 - protein, 13 aa, Artificial Sequence
  Description of Artificial Sequence Synthetic peptide
QNILLSNAPLGPQ

SEQ ID NO: 27 - protein, 13 aa, Artificial Sequence
  Description of Artificial Sequence Synthetic peptide
SYLQDSDPDSFQD

SEQ ID NO: 28 - protein, 9 aa, Artificial Sequence
  Description of Artificial Sequence Synthetic peptide
SYLDSGIHF

SEQ ID NO: 29 - protein, 9 aa, Artificial Sequence
  Description of Artificial Sequence Synthetic peptide
EEKLIVVLF

SEQ ID NO: 30 - protein, 10 aa, Artificial Sequence
  Description of Artificial Sequence Synthetic peptide
ACDPHSGHFV

SEQ ID NO: 31 - protein, 25 aa, Porphyromonas gingivalis
GVSPKVCKDVTVEGSNEFAPVQNLT

SEQ ID NO: 32 - protein, 77 aa, Homo sapiens
RGVDCLSSHFQELSIYQDQEQRILKFLEELGEGKATTAHDLSGKLGTPKKEINRVLYSLAKKGKLQKEAGTPPLWKI

(19) United States
(12) Patent Application Publication
(10) Pub. No.: US 2011/0142874 A1
(43) Pub. Date: Jun. 16, 2011

(54) Compositions, Methods, and Kits for Eliciting an Immune Response

(75) Inventors: Bertram Jacobs, Tempe, AZ (US); Karen Kibler, Scottsdale, AZ (US); Trung Huynh, Tempe, AZ (US)

(73) Assignee: Arizona Board of Regents, Scottsdale, AZ (US)

(21) Appl. No.: 12/996,942

(22) PCT Filed: Jun. 9, 2009
(86) PCT No.: PCT/US09/46790

§ 371 Date: Feb. 22, 2011

Related U.S. Application Data
(60) Provisional application No. 61/059,990, filed on Jun. 9, 2008.

(51) Int. Cl. A61K 39/385; C07K 14/00; C07H 21/02; C07H 21/04; C12N 5/00

(52) U.S. Cl. 424/193.1; 530/350; 536/23.4; 435/325

(57) ABSTRACT

The present invention relates to compositions, methods, and kits for eliciting an immune response to at least one antigen, in particular for enhancing antigen immunogenicity.

### RELATED APPLICATIONS

This application claims priority to U.S. provisional application 61/059,990, which was filed on Jun. 9, 2008, which is herein incorporated in its entirety.

### GOVERNMENT INTERESTS

The development of this invention was made with Government support under grant number AI52347 awarded by the National Institutes of Health. The Government has certain rights in the invention.

### FIELD OF THE INVENTION

The present invention relates to novel compositions, methods, and kits for eliciting an immune response against an antigen.

### BACKGROUND OF THE INVENTION

Conventional methods of immunological protection against disease by vaccination involves the administration of a disease-associated antigen that will elicit an immune response against the antigen, so that when challenged later with the antigen, the vaccinated individual is protected against the disease.

Immunogenicity of an antigen can be improved by the addition of an adjuvant. In various cases, it has been reported that materials that have little or no immunogenicity have been made to elicit high titres of antibody in vivo by the addition of an adjuvant, however, some of these adjuvants can have undesirable side effects. For example, it has been suggested that double-stranded nucleic acids can act through TLR3, RIG-1, MDA5, and other immunity signaling molecules to boost immune responses. However, previous methods of providing double-stranded nucleic acids as adjuvants have had undesirable effects including toxic effects.

Thus, there is a need for the development of effective, less toxic strategies that elicit an immune response to an antigen.

### SUMMARY OF THE INVENTION

The present invention provides a conjugate comprising at least one antigen or small molecule conjugated to at least one polypeptide capable of binding a double-stranded polynucleotide. In certain embodiments, the double-stranded polynucleotide comprises a double-stranded polyribonucleotide (dsRNA).

In certain embodiments of the invention, the at least one polypeptide comprises a binding domain of a protein selected from the group consisting of: ADAR1, ZBP1, PKR-like kinase, and E3L. In certain embodiments, the at least one polypeptide comprises a Z-alpha domain or a variant thereof.

In other embodiments, the double stranded polyribonucleotide (dsRNA) comprises a first ligand. In certain embodiments, the conjugate also comprises a second ligand capable of binding to the first ligand. The second ligand may be a multimer, capable of binding more than one ligand. In certain embodiments the first ligand is biotin and the second ligand comprises a tetrameric avidin. Other adjuvants and/or TLR agonists having a third ligand attached thereto may also be included in the conjugate. The third ligand on the adjuvant and/or the TLR agonist will bind the second ligand. The second ligand (i.e. tetrameric avidin) in turn binds the first ligand (biotin) on the dsRNA.

In some embodiments, the first ligand comprises biotin and the second ligand comprises a tetrameric avidin, and the adjuvant and/or the TLR agonist comprise a third ligand, biotin.

In some embodiments, the at least one TLR agonist is selected from the group consisting of a TLR 4 agonist, a TLR9 agonist, a TLR 5 agonist, and a TLR 7/8 agonist. In certain embodiments the TLR agonist is selected from the group consisting of LPS, unmethylated CpG DNA, resiquimod and flagellin.

The present invention also provides compositions comprising a conjugate of the invention and a pharmaceutically acceptable carrier. The composition may further comprise a double-stranded polynucleotide, such as dsRNA. The double-stranded polynucleotide may comprise a first ligand. The composition may comprise a second ligand, preferably in a multermeric form that can bind the first ligand and additional adjuvants and/or TLR agonists having a third ligand that recognizes and binds the multimeric second ligand. The composition may be useful as a vaccine to elicit an immune response to an antigen or small molecule.

The present invention also provides a DNA or RNA molecule encoding a conjugate of the invention or a host cell comprising the DNA or RNA molecule encoding a conjugate of the invention.

The present invention also provides a method for eliciting an immune response to an antigen or small molecule in a subject, the method comprises administering to the subject a conjugate or composition of the present invention.

In some embodiments the small molecule comprises cocaine and the polypeptide comprises E3L. In certain embodiments, the small molecule comprises cocaine, and the polypeptide comprises E3L and the TLR agonist comprises unmethylated CPG DNA.

The present invention also provides a kit comprising a polypeptide capable of binding a double-stranded polynucleotide and optionally contains a dsRNA labeled with a first ligand, such as biotin. The kit may optionally comprise a second ligand, and is preferably an avidin mulimer. The kit may further comprise additional adjuvants or TLR agonists having a third ligand, wherein the third ligand is capable of binding the second ligand.

### BRIEF DESCRIPTION OF THE FIGURES

FIG. 1 shows an embodiment of the invention.

FIG. 2 shows an embodiment of the invention—norococaine-GST-E3L-dsRNA nanoparticles.

FIG. 3 shows the structure of imiquimod and an imiquimod-biotin analogue used in an embodiment of the invention.

FIG. 4 shows an exemplary synthesis scheme of an imiquimod-biotin analogue.

FIG. 5 shows an exemplary synthesis scheme of a resiquimod-biotin analogue.

FIG. 6 shows an exemplary synthesis scheme of a six carbon tether.

FIG. 7 shows cocaine haptenes that can be used to form cocaine immunoconjugates.

FIG. 8 shows a synthesis scheme of compound 12 (a cocaine hapten).

FIG. 9 shows binding of E. coli expressed GST-E3L to in vitro synthesized PKR.

FIG. 10 shows binding of E. coli expressed purified GST-E3L to in vitro synthesized dsRNA.

### DETAILED DESCRIPTION OF THE INVENTION

The present invention provides compositions and methods for eliciting an immune response to at least one antigen or small molecule of interest, in particular to enhance immunogenicity of the at least on antigen/small molecule. In accordance with the present invention, the at least one antigen/small molecule of interest is conjugated to at least one polypeptide capable of binding double-stranded nucleic acids, preferably double stranded RNA (dsRNA). Since the conjugate is capable of binding the double-stranded nucleic acid, it provides novel adjuvanted antigens/small molecules.

In certain embodiments the at least one polypeptide comprises a binding domain of a protein selected from the group consisting of: ADAR1, ZBP1, PKR-like kinase, and E3L. E3L protein binds to double stranded RNA. dsRNA is a potent PAMP, TLR3 ligand and adjuvant and sequestration of dsRNA by E3L protein inhibits pro-inflammatory signal transduction and pro-inflammatory gene expression. But this inhibition of pro-inflammatory signaling comes at a cost: E3L protein is itself a potent antigen. Without being bound by theory, it is believed that E3L protein is a potent antigen because it is presented to the immune system bound to a very powerful PAMP, dsRNA. Thus, the fusion of any antigen of interest to E3L protein should increase immunogenicity of the fused protein, because now the antigen of interest would be presented to the immune system bound to the PAMP and adjuvant, dsRNA. Thus, the antigen would carry its own adjuvant into immune cells. This would not only decrease the amount of adjuvant seen by the body, which would likely decrease any toxicity associated with the adjuvant, but would increase the local concentration of the adjuvant in the cells actually presenting the antigen.

The past decade has seen an explosion of information on how our innate immune system recognizes pathogens and stimulates induction of adaptive immunity (28). Dozens of toll like receptors (TLRs), Rig-like receptors (RLRs) and NOD-like receptors (NLRB) (as a group known as Pathogen Recognition Receptors, or PRRs), recognize pathogen associated molecular patterns (PAMPs) and induce pro-inflammatory signaling, pro-inflammatory gene expression (4, 16, 28) and maturation of antigen presenting cells (4). Thus, recognition of PAMPs by PRRs can lead to induction of potent immune responses. For one of the most potent immunogens, the yellow fever 17D vaccine, enhanced immunogenicity appears to be associated with signaling through multiple PRRs (6). Thus, presentation of antigen bound to multiple PAMPs may lead to induction of robust and long-lived immunity.

One particularly potent PAMP is double-stranded RNA (dsRNA). DsRNA can lead to signaling through TLR3, RLRs, and NALP3 (5). DsRNA can lead to induction of pro-inflammatory signaling, and induction of pro-inflammatory gene expression, including induction of type I and III interferons (16).

In one embodiment, the antigen-polypeptide conjugate is used in the context of a live viral vector, where the antigen of interest is fused to polypeptide (such as E3L), for instance in a vaccinia virus vector, like NYVAC or ALVAC II. The virus vector is provided to an individual and is expressed within the individual to produce a fusion protein of the antigen-polypeptide.

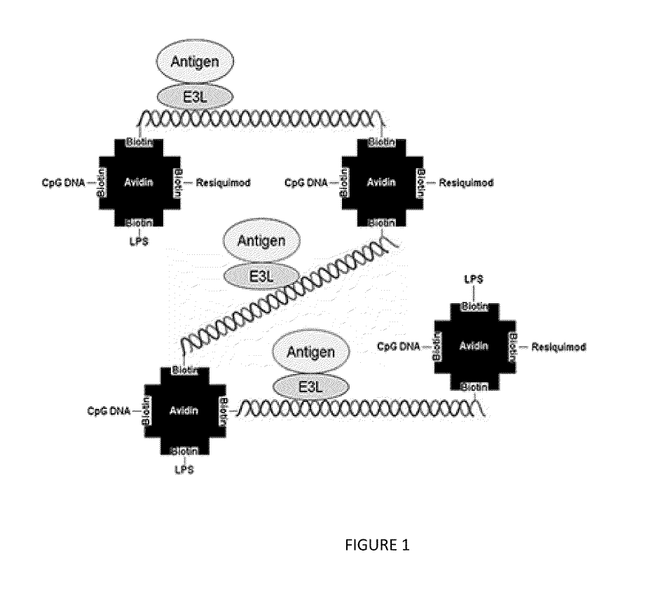

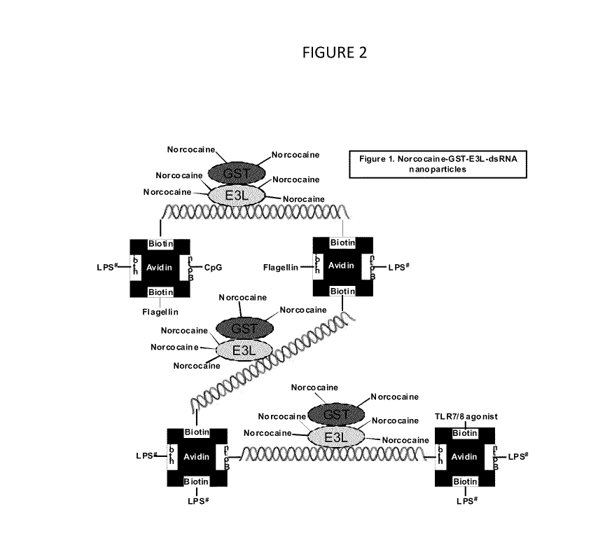

Alternatively, the antigen/small molecule-polypeptide fusion protein could be synthesized in prokaryotic or eukaryotic cells, purified, loaded with synthetic dsRNA and used as a soluble antigen. In certain embodiments, the antigen/small molecule-polypeptide conjugate is haptenized. Methods of haptenizing antigens and small molecules are known in the art. This approach is in many ways very attractive; because it allows the use of synthetic biology to tailor make the adjuvant for the antigen of interest. For instance the dsRNA used to load onto the antigen/small molecule-polypeptide fusion protein conjugate can be synthesized to contain a first ligand. Then a second ligand that is able to bind to the first ligand on the dsRNA can be constructed and added to the antigen-polypeptide fusion protein conjugate. The second ligand may be in a multimeric form so that other adjuvants may be constructed to contain a third ligand so that they bind to the multimeric second ligand. For example, an antigen/small molecule-E3L fusion protein can be loaded with synthetic dsRNA that is biotinylated (biotin as the first ligand). Then a multermic form of a second ligand (such as a tetrameric avidin) is added to the biotinylated dsRNA (avidin as the second ligand). Then any other adjuvants and/or TLR ligands (or combinations thereof) can be made to contain a third ligand (i.e. biotin) and thus can be loaded onto the multimeric avidin complex. See FIG. 1 for an antigen-E3L fusion protein and FIG. 2 for a small molecule-E3L fusion protein.

For instance, the fusion protein/dsRNA/biotin/avidin complex could be incubated with biotinylated-LPS (a commercially available TLR4 agonist), biotinylated unmethylated CpG DNA (a commercially available TLR9 agonist), biotinylated flagellin (a TLR5 agonist) or biotinylated resiquimod, a TLR7/8 agonist (while biotinylated resiquimod is not commercially available, methods for conjugating resiquimod to other ligands without modifying its adjuvant activity have been described). Further, TLR 7/8 agonist have been shown to increase immunogenicity of soluble antigens (12).

A further advantage of this technology is that depending on the molar ratio of compounds added to the mixture, aggregates of varying sizes would be expected. Since aggregation can itself increase immunogenicity of soluble antigens, this could in itself act as an adjuvant.

The present invention thus provides many different combinations of adjuvants or TLR agonists bound to dsRNA through ligand-ligand interactions, such as with biotin-avidin, of many different average sizes. Then for any new antigen or small molecule, the conjugate can be made as a fusion of the antigen/small molecule to E3L, loaded onto the pre-existing adjuvant scaffolds and tested for immunogenicity. This would allow a very rapid testing of “adjuvant space,” allowing identification of the best combination of adjuvants for use for any given antigen/small molecule.

While others have shown that antigen bound to a TRL agonist, reisquimod, increases immunogenicity, this was done by chemical modification of the antigen, which is a labor intensive, antigen-by-antigen procedure. The present invention overcomes this problem, by expressing the antigen fused to a TLR ligand binding domain or an adjuvant. This potentially offers a wider utility than chemical modification of antigens. Furthermore, the technology described in this application can load many different TLR ligands or antagonists onto an antigen/small molecule.

In embodiments of the invention where a small molecule (instead of an antigen) is conjugated to at least one polypeptide capable of binding double-stranded nucleic acids, (i.e. dsRNA), the small molecule is preferably haptenized to a protein to be able to induce antibodies.

In one embodiment, the small molecule is cocaine. Cocaine usage inflicts a huge cost on society. It is estimated that there are over 2 million cocaine abusers in the US (13, 19). Cocaine accounts for 30 to 40% of all emergency room visits related to illegal drug use. Nearly 30% of US prisoners were regular users of cocaine prior to incarceration. At any one time there are estimated to be between one and 3 million people in need of treatment for cocaine abuse (19). Yet no effective treatment for cocaine abuse exists. Standard drug counseling alone appears to have minimal impact on cocaine relapse (19), and no effective pharmacological intervention exists (2). Thus, there is a great need for an effective intervention for cocaine abuse.

Therapeutic vaccines for cocaine, and other drugs of abuse, hold much promise (15, 22). If IgG concentrations to cocaine in the blood are high enough and if antibodies have a high enough avidity for cocaine, then it is likely that free cocaine levels in the blood can be drastically reduced (22). Since IgG does not generally cross the blood brain barrier, binding of cocaine to anti-cocaine IgG can potentially reduce the amount of drug that can reach the brain. Thus, the greatest potential benefit of an anti-cocaine vaccine would be to prevent the reinforcing effects of cocaine re-exposure in patients who are attempting to stop cocaine use (22). Reducing the pleasurable effects of occasional cocaine re-exposure, and reducing the subsequent craving responses, could help greatly in preventing motivated users from succumbing to the effects of occasional use brought on by specific social or emotional circumstances.

Since cocaine is a small molecule it must first be haptenized to a protein in order to induce antibodies to cocaine (17). Norcocaine derivatives have been successfully used to haptenize KLH and CTB (9, 19). Proteins have also been haptenized with cocaines derivatized at the ester group of cocaine (2). Administration of these haptenized proteins using alum or RIBI as an adjuvant can induce antibodies to cocaine in both experimental animals and in humans (2, 19). In rats, immunization significantly decreased response to cocaine (2) and in humans, higher levels of antibodies to cocaine correlated with reduced cocaine usage (19). Nonetheless, antibody levels induced in humans by vaccination were 20-fold less than levels required to block cocaine activity in experimental animals (19). Thus, while the initial results of clinical trials have been promising, a vaccine which induces higher levels of antibodies in humans is needed.

Vaccine formulations tested so far both in experimental animals and in humans have been fairly conservative, consisting of haptenized proteins generally administered in alum or RIBI (2, 19) (there has been a single report of using an alternative adjuvant (9)). However, no studies have yet been published that take advantage of the recent revolution in the understanding of the role of innate immunity in induction of an antibody response. Nor have any studies been published that take advantage of nanoparticles in cocaine immunogen design.

### I. DEFINITIONS

The term “conjugate” herein refers to molecules (e.g., polypeptides) that are joined together. The molecules can be joined together by various means including, but not limited to covalent bonding or affinity bonding. For example, a “conjugate” can refer to a single fusion polypeptide produced recombinantly from a cDNA having at least two operatively linked heterologous gene sequences fused in the correct reading frame so that a recombinant fusion protein (i.e., conjugate) is expressed. By way of another example, a “conjugate” can refer to a complex based on non-covalent bonding (e.g., a first polypeptide comprising biotin joined to a second polypeptide comprising streptavidin via biotin/streptavidin interaction). Accordingly, as used herein, the terms “conjugate, “conjugated,” “fusion,” or “fused” broadly refer to joined, and includes joining by any method, including but not limited to, covalent and non-covalent methods, and permanently to non-permanently.

The term “polypeptide” refers to a polymer of amino acids and does not refer to a specific length of the product; thus, peptides, oligopeptides, and proteins are included within the definition of polypeptide. This term also does not refer to or exclude post-expression modifications of the polypeptide, for example, glycosylations, acetylations, phosphorylations and the like. Included within the definition are, for example, polypeptides containing one or more analogues of an amino acid (including, for example, unnatural amino acids, etc.), polypeptides with substituted linkages, as well as other modifications known in the art, both naturally occurring and non-naturally occurring.

The term “variant” of a polypeptide refers to an amino acid sequence that is altered by one or more amino acids. The variant can have “conservative” changes, wherein a substituted amino acid has similar structural or chemical properties, e.g., replacement of leucine with isoleucine. Alternatively, a variant can have “non-conservative” changes, e.g., replacement of a glycine with a tryptophan. Analogous minor variation can also include amino acid deletion or insertion, or both. A particular form of a “variant” polypeptide is a “functionally equivalent” polypeptide, i.e., a polypeptide which exhibits substantially similar in vivo or in vitro activity as the examples of the polypeptide of invention, as described in more detail below. Guidance in determining which amino acid residues can be substituted, inserted, or deleted without eliminating biological or immunological activity can be found using computer programs well-known in the art, for example, DNASTAR software (DNASTAR, Inc., Madison, Wis.).

The term “double-stranded polynucleotide” refers herein to a nucleic acid molecule comprising a region having two or more nucleotides that are in a double-stranded conformation.

### II. CONJUGATE

### The at Least One Antigen

In one aspect, the present invention provides a conjugate comprising at least one antigen conjugated to at least one polypeptide capable of binding a double-stranded polynucleotide.

The at least one antigen can correspond to any polypeptide against which an immune response is to be elicited. Generally, the at least one antigen comprises sufficient structure (e.g., primary, secondary, tertiary, or quaternary structure) to be recognized by T and/or B cells, and the antibodies secreted by B cells. For example, the at least one antigen can comprise a sufficient number of amino acid residues corresponding to the portion of an antigenic protein that functions as an antigenic determinant to induce a cell-mediated or humoral immune response, i.e., either a T cell or B cell epitope, or both. Accordingly, in some embodiments, the at least one antigen comprises at least about 4 amino acid residues corresponding to an epitope.

The at least one antigen can be any type of antigen. In one embodiment, the at least one antigen is a disease-associated antigen having at least one epitope. The disease-associated antigen can be characterized as an antigen that is selectively expressed by a diseased cell, or it can be characterized as an antigen expressed by both diseased and normal cells.

The antigen can be derived from any suitable source, for example from bacteria, viruses, plasmodium, flat worms or round worms, etc.

The at least one antigen can be a tumor-associated antigen. The tumor-associated antigen can be tumor-specific, tumor-selective, or both. Tumor-associated antigens include but are not limited to antigens corresponding to p53, Ras, Bcr/Abl breakpoint peptides, HER-2/Neu, HPV E6, HPV E7, carcinoembryonic antigen, MUC-1, MAGE-1, MAGE-3, BAGE, GAGE-1, GAGE-2, N-acetylglucosaminyltransferase-V, p15, gp100, MART-1/MelanA, tyrosinase, TRP-1, β-catenin, MUM-1, CDK-4, and mutants thereof.

In one embodiment, the tumor-associated antigen can be an oncogenic protein such as a nonmutated, overexpressed oncoprotein or a mutated, unique oncoprotein. For example, mutations in p53 are present in about 50% of human malignancies, and a mutant p53 protein or peptide fragment thereof can be a tumor-associated antigen useful in the present invention. A tumor-associated antigen can also be a normal p53 protein or peptide fragment thereof, wherein a selective immune response against tumor cells is achieved due to the relative increased accumulation of p53 in the cytosol of tumor cells.

Mutant Ras proteins and peptide fragments thereof can also be tumor-associated. Mutant Ras proteins can have a single amino acid substitution at residue 12 or 61, for example, and Ras peptides spanning this mutant segment can be useful tumor-associated antigens.

HER-2/neu can also be a tumor-associated antigen, and peptides derived from the HER-2/neu proto-oncogene can be useful in the compositions and methods of the present invention.

In another embodiment, the tumor-associated antigen can be the epidermal growth factor receptor (EGFR) or immunogenic epitope thereof, or a mutant EGFR variant or immunogenic epitope thereof. For example, the EGFR deletion mutant EGFRvIII is expressed in a subset of breast carcinomas and in non-small cell lung carcinomas and malignant gliomas. EGFRvIII disease-associated antigens, such as peptides corresponding to the EGFRvIII fusion junction, can be useful in stimulating an immune response against such tumors. Thus, EGFR or EGFRvIII disease-associated antigens or immunogenic epitopes thereof can be useful for the treatment of breast and lung carcinomas and malignant gliomas and to protect individuals at high risk from developing these cancers.

The tumor-associated antigen can also be an E6 or E7 viral oncogene such as a human papilloma virus (HPV) E6 or E7 viral oncogene or immunogenic epitope thereof. For example, HPV16 is one of the major human papillomavirus types associated with cervical cancer, and immunogenic peptide epitopes encoded by HPV16 E6 and E7 can be useful for the prevention and treatment of cervical carcinoma.

The tumor-associated antigen can also be carcinoembryonic antigen (CEA), which is an antigen that is highly expressed in the majority of colorectal, gastric, and pancreatic carcinomas.

The MUC-1 mucin gene product, which is an integral membrane glycoprotein present on epithelial cells, also is a tumor-associated antigen useful in the present invention. Mucin is expressed on human epithelial cell adenocarcinomas, including breast, ovarian, pancreatic, lung, urinary bladder, prostate and endometrial carcinomas, presenting more than half of all human tumors. Compositions and methods of the present invention containing full-length mucin or immunogenic epitopes thereof can therefore be used to protect against or treat epithelial cell adenocarcinomas such as breast carcinomas.

Minor histocompatibility antigens (e.g., HLA-A2 antigen) can also be used as tumor-associated antigens in accordance with the present invention.

A number of melanoma antigens also are characterized as tumor-associated antigens. For example, the MAGE-1, MAGE-2, MAGE-3, BAGE, GAGE-1, and GAGE-2 tumor-associated antigens or immunogenic epitopes thereof such as MZ2-E can be used. Melanoma tumor-associated antigens can also be differentiation antigens expressed by normal melanocytes. Such melanoma tumor-associated antigens include MART-1/MelanA; gp100; tyrosinase; and tyrosinase-related protein TRP-1 (gp75).

Exemplary disease-associated antigens and corresponding exemplary epitopes include, but are not limited to, HER-2/neu (e.g., IISAVVGIL, KIFGSLAFL); HPV E6, HPV E7 (e.g., YMLDLQPETT), MUC-1 (e.g., PDTRPAPGSTAPPA, HGVTSA); MAGE-1 (e.g., EADPTGHSY, SAYGEPRKL); MAGE-3 (e.g., EVDPIGHLY, FLWGPRALV); BAGE (e.g., AARAVFLAL); GAGE-1, GAGE-2 (e.g., YRPRPRRY); GnT-V (e.g., VLPDVFIRC); p15 (e.g., AYGLDFYIL); gp100 (e.g., KTWGQYWQV, ITDQVPFSV, YLEPGPVTA, LLDGTATLRL, VLYRYGSFSV); MART-1/MelanA (e.g., AAGIGILTV, ILTVILGVL); TRP-1 (e.g., MSLQRQFLR); Tyro-sinase (e.g., MLLAVLYCL, YMNGTMSQV, SEIWRDIDF, AFLPWHRLF, QNILLSNAPLGPQ, SYLQDSDPDSFQD); β-catenin (e.g., SYLDSGIHF); MUM-1 (e.g., EEKLIVVLF); and CDK4 (e.g., ACDPHSGHFV).

In one embodiment, the at least one antigen comprises an epitope having an amino acid sequence selected from the group consisting of: IISAVVGIL, KIFGSLAFL, YMLDLQPETT, PDTRPAPGSTAPPA, HGVTSA, EADPTGHSY, SAYGEPRKL, EVDPIGHLY, FLWGPRALV, AARAVFLAL, YRPRPRRY, VLPDVFIRC, AYGLDFYIL, KTWGQYWQV, ITDQVPFSV, YLEPGPVTA, LLDGTATLRL, VLYRYGSFSV, AAGIGILTV, ILTVILGVL, MSLQRQFLR, MLLAVLYCL, YMNGTMSQV, SEIWRDIDF, AFLPWHRLF, QNILLSNAPLGPQ, SYLQDSDPDSFQD, SYLDSGIHF, EEKLIVVLF, and ACDPHSGHFV.

A disease-associated antigen can also be a human immunodeficiency type I (HIV-1) antigen, for example the gp120 envelope glycoprotein and immunogenic epitopes thereof such as the principal neutralization determinant (PND); gp160; and HIV-1 core protein derived immunogenic epitopes.

A disease-associated antigen of the present invention can also contain autoimmune disease-associated antigens corresponding to such diseases as rheumatoid arthritis, psoriasis, multiple sclerosis, systemic lupus erythematosus and Hashimoto's disease, type I diabetes mellitus, myasthenia gravis, Addison's disease, autoimmune gastritis, Graves' disease and vitiligo. Autoimmune disease-associated antigens can be, for example, T cell receptor derived peptides such as Vβ14, Vβ3, Vβ17, Vβ13 and Vβ6 derived peptides. Autoimmune disease-associated antigens can also include annexins such as AX-1, AX-2, AX-3, AX-4, AX-4, AX-5 and AX-6, which are autoantigens associated with autoimmune diseases such as systemic lupus erythematosus, rheumatoid arthritis and inflammatory bowel disease.

A number of other disease-associated antigens also can be included in the present invention such as, for example, viral, parasitic, yeast, and bacterial antigens. For example, Helicobacter pylori is the major causative agent of superficial gastritis. and plays a central role in the etiology of peptic ulcer disease. In this embodiment, the disease-associated antigen can be, for example, the urease protein, 90 kDa vacuolating cytotoxin (VacA), or 120 to 140 kDa immunodominant protein (CagA) of H. pylori, or immunogenic epitopes thereof.

Disease-associated antigens derived from P. gingivalis also can be included in the present invention. P. gingivalis disease-associated antigens include the ArgI, ArgIA and ArgIB arginine-specific proteases of P. gingivalis, and immunogenic epitopes thereof including the GVSPKVCKDVTVEGSNEFAPVQNLT epitope.

In other embodiments, the disease-associated antigens can be selected from the MP65 antigen of Candida albicans; helminth antigens; Mycobacterial antigens including M. bovis and M. tuberculosis antigens; Haemophilus antigens; Pertussis antigens; cholera antigens; malaria antigens; influenza virus antigens; respiratory syncytial viral antigens; hepatitis B antigens; poliovirus antigens; herpes simplex virus antigens; rotavirus antigens, and flavivirus antigens.

### Small Molecules

Any small molecule for which a vaccine would be useful may be used in the present invention. One exemplary molecule comprises cocaine.

### The at Least One Polypeptide

The at least one polypeptide capable of binding a double-stranded polynucleotide can be a full-length protein (e.g., ADAR1) or a fragment thereof (e.g., z-alpha domain of ADRA1) comprising the domain(s) capable of binding a double-stranded polynucleotide. Variants of the full-length protein or binding fragments thereof also are within the scope of the present invention.

For example, in some embodiments of the present invention, the at least one polypeptide comprises one or more double-stranded polynucleotide binding domains of a protein, wherein the one or more domains are capable of binding double-stranded polynucleotides. Binding of the double-stranded polynucleotide can be either nucleic acid sequence-specific and/or independent of nucleic acid sequence.

A number of methods are available to determine, if necessary, a minimum number of amino acid residues required to form a functional double-stranded polynucleotide binding polypeptide. For example, a double-stranded polynucleotide binding protein can be digested, for example with an endoprotease, to generate polypeptide fragments, which can be isolated, and determined (e.g., using band-shift assays known in the art) for their ability to bind double-stranded polynucleotide.

Nucleic acid binding domains also can be determined by homology assessment with known sequences corresponding to known double-stranded polynucleotide binding proteins, or can be determined using biochemical methods, for example. On example of homology searching is the BLAST algorithm, which performs a statistical analysis of the similarity between two sequences (see, e.g., Karlin & Altschul, PNAS, 90:5873 (1993)). One measure of similarity provided by the BLAST algorithm is the smallest sum probability (P(N)), which provides an indication of the probability by which a match between two nucleotide or amino acid sequences would occur by chance.

For example, domains that have at least about 50% amino acid sequence identity, illustratively, about 50, 60, 75, 80, 85, 90, 92, 94, 96, 98, 99, 99.5% amino acid sequence identity to a known sequence of a double-stranded polynucleotide binding protein or binding fragment thereof over a comparison window of at least about 25 amino acids, optionally about 50-100 amino acids, or the length of the entire protein, can be used in the invention. The sequence can be compared and aligned for maximum correspondence over a comparison window, or designated region as measured using one of the following sequence comparison algorithms or by manual alignment and visual inspection. Percent amino acid identity can be determined by the default parameters of BLAST, for example.

In some embodiments, the at least one polypeptide is capable of binding a double-stranded polynucleotide, wherein the double-stranded polynucleotide comprises RNA, DNA, or both. Polypeptides capable of binding RNA/DNA duplexes are also within the scope of the present invention. It will be understood by one of ordinary skill in the art that although the at least one polypeptide of the present invention is capable of binding a double-stranded polynucleotide, the at least one polypeptide also may be further characterized as capable of binding other forms of nucleic acids including single-stranded forms. Thus, for example, in some embodiments, the at least one polypeptide can be characterized as capable of binding a double-stranded polynucleotide at about the same, about 2-fold, 4-fold, 6-fold, 8-fold, 10-fold, 100-fold, 1000-fold, 10.000-fold, or greater affinity than a single-stranded nucleic acid.

Generally, binding of the at least one polypeptide to a double-stranded polynucleotide can be qualitatively and/or quantitatively assessed, if desired, by methods known in the art. For example, a competition analysis can be performed wherein binding of labeled double-stranded polynucleotide is competed by the addition of increasing amounts of unlabelled nucleic acid. Alternatively, binding can be assessed by a gel shift assay in which labeled double-stranded polynucleotide is incubated with the at least one polypeptide. The protein-nucleic acid complex will migrate slower through the gel than unbound nucleic acid, resulting in a shifted band. The amount of binding can be assessed by incubating samples with increasing amounts of double-stranded, and quantifying the amount of radioactivity in the shifted band. Such qualitative and/or quantitative assessments also can be analyzed based on relative binding of the at least one polypeptide to single-stranded nucleic acids.

A number of eukaryotic, prokaryotic, and viral polypeptides or fragments thereof are known in the art that can bind double-stranded polynucleotide. Illustrative non-limiting examples of double-stranded polynucleotide binding polypeptides include polypeptides, or fragments thereof, disclosed by e.g., GENBANK Accession Numbers: CAH71908.1; NP_{—}001102.2; P55265.2; DSRAD_HUMAN; NP_{—}056655.2; AAB97116.1; CAA55968.1; CAA67170.1; XP_{—}513841.2; BAD93128.1; NP_{—}056656.2; AAB97117.1; CAD98075.1; CAE45853.1; 1QBJ; 1QGP; ABM73522.1; XP_{—}001111902.1; 2ACJ; XP_{—}581374.3; XP_{—}547564.2; XP_{—}001497601.1; ABM73521.1; XP_{—}001373191.1; EDM00617.1; NP_{—}112268.1; AAK16102.1; Q99MU3; AAC06233.1; BAC40888.2; NP_{—}062629.2; AAK17103.1; EDL15185.1; AAS82589.1; CAJ18531.1; XP_{—}001518190.1; NP_{—}571671.1; AAB51688.1; AAH44344.1; NP_{—}001081675.1; NP_{—}957929.1; XP_{—}001183590.1; AAF69764.1; XP_{—}789034.2; AAF69674.1; NP_{—}659606.1; AAQ18045.1; YP_{—}156772.1; NP_{—}001117067.1; AAP49830.1; YP_{—}001497028.1; NP_{—}150468.1; YP_{—}227418.1; YP_{—}001293225.1; AAN02759.1; NP_{—}570192.1; ZP_{—}01446883.1; AAQ18046.1; 1SFU; BAF48125.1; CAG11855.1; XP_{—}854632.1; NP_{—}073419.1; ABB84392.1; XP_{—}342595.1; Q8VDA5.1; 2HEO; ZP_{—}02131943.1; 1XMK; NP_{—}069051.1; ABI99027.1; NP_{—}671561.1; XP_{—}001114247.1; YP 001753747.1; AAO32333.1; AAA02759.1; ABD52517.1; ABF73314.1; AAS49761.1; AAP43510.1; YP_{—}232941.1; AAQ18044.1; AAQ18043.1; EAW53188.1; EAW53184.1; CAA10953.1; 1OYI; AAQ94311.1; CAA10952.1; CAA55967.1; CAA67169.1; NP_{—}001020278.1; AAC08018.1; AAQ94312.1; ABB84393.1; ABB84391.1; ABB84395.1; XP_{—}001170447.1; ABB84394.1; ABB84398.1; AAW23462.1; P21081; P55266; AAK16102; AAB51687; AF051275; P78563; P51400; AAK17102; AAF63702; AAF78094; AAB41862; AAF76894; AAA36409; AAA61926; Q03963; AAA36765; P97473; AAC25672; AAD33098; AAA49947; NP_{—}609646; AAD17531; AAF98119; AAD17529; P25159; AF167569; AF167570; AAF31446; AAC71052; AAA19960; AAA19961; AAG22859; AAK20832; AAF59924; A57284; CAA71668; AAC05725; AAF57297; AAK07692; AAF23120; AAF54409; T33856; AAK29177; AAB88191; AAF55582; NP_{—}499172; NP_{—}198700; BAB19354; NP_{—}563850; CAC05659; BAB00641; XP_{—}059592; CAA59168; AAF80558; AAF59169; Z81070; Q02555/S55784; P05797; BAA78691; AF408401; AAF56056; 544849; AAF03534; Q09884; and AY071926, each of which is incorporated herein by reference in its entirety.

Examples of nucleic acid binding proteins also are described in e.g., Saunders et al., FASEB, 17:961 (2003); Brown et al., PNAS, 97:13532 (2000); Kwon et al., PNAS, 102:12759 (2005); Wilsker et al., Cell Growth & Differentiation, 13:95 (2002); Schwartz et al., Nat. Struct. Biol., 8:761 (2001); Deigendesch et al., Nucleic Acids Research, 34:5007 (2006); Herbert et al., Nucleic Acids Res., 26:3486 (1998); and U.S. Pat. Nos. 5,858,675; 5,843,643, and 6,627,424, each of which is incorporated herein by reference for its teaching of double-stranded polynucleotide binding polypeptides or fragments thereof.

By way of another example, the human ADAR1 z-alpha domain having the amino acid sequence corresponding to residues 121-197 as shown in GENBANK Accession No. CAH71908 can bind to left-handed Z-DNA as well as Z-RNA (See e.g., Brown et al., PNAS, 97:13532 (2000); and Schwartz et al., JBC, 274:2899 (1999)). The z-alpha domain of human ADAR1 belongs to a z-alpha domain superfamily, which also includes but is not limited to z-alpha domains from the tumor-related DLM1 (or ZBP1) protein, PKR-like kinase of bony fish, and E3L protein of vaccinia virus. Accordingly, in one embodiment, the at least one polypeptide comprises an amino acid sequence corresponding to a z-alpha domain or a variant thereof. In another embodiment, the z-alpha domain is a protein selected from the group consisting of: ADAR1, ZBP1, PKR-like kinase, and E3L.

In other embodiments, the z-alpha domain comprises the amino acid sequence: RGVDCLSSHFQELSIYQDQEQRILKFLEELGEGKATTAHDLSGKLGTPKKEINRVLYSLA KKGKLQKEAGTPPLWKI or a variant thereof.

Other examples of polypeptides that are capable of binding a double-stranded polynucleotide include polymerases, exonucleases, reverse transcriptases, methylases, ligases, restriction endonucleases, gyrases, topoisomerases, and polyamides. Polyamides, which comprise polymers of amino acids covalently linked by amide bonds, are described in e.g., U.S. Pat. Nos. 6,143,901; 6,090,947; and 6,635,417, each of which is described herein by reference for its teaching of polyamides.

### Conjugating

The at least one antigen and the at least one polypeptide can be joined to form the conjugate by methods known to those of skill in the art, which methods can include but are not limited to chemical and recombinant methods.

Chemical methods of joining heterologous polypeptides are described, e.g., in Bioconjugate Techniques, Hermanson, Ed., Academic Press (1996). These include, for example, derivitization for the purpose of linking moieties to each other, either directly or through a linking compound, by methods that are well known in the art of protein chemistry.

For example, in one chemical conjugation embodiment, the method of linking the at least one antigen and the at least one polypeptide comprises a heterobifunctional coupling reagent which ultimately contributes to formation of an intermolecular disulfide bond between the two moieties. Alternatively, an intermolecular disulfide can be formed between cysteines in each moiety, which occur naturally or are inserted by genetic engineering. The methods of linking moieties can also use thioether linkages.

Examples of linking agents include but are not limited to chemical cross-linking agents such as, for example, succinimidyl-(N-maleimidomethyl)-cyclohexane-1-carboxylate (SMCC). The linking group can also be an additional amino acid sequence(s), including, for example, a polyalanine, polyglycine, and the like.

The methods of linking polypeptides can also comprise a peptidyl bond formed between moieties that are separately synthesized by standard peptide synthesis chemistry or recombinant methods. For example, peptides can be synthesized by solid phase techniques, wherein amino acids are sequentially added to a growing chain of amino acids. Optionally, amino acids analogs can be introduced as a substitution or insertion into the sequence.

In one embodiment, the coding sequences of two or more polypeptides in the conjugate are directly joined at their amino- or carboxy-terminus via a peptide bond in any order. Alternatively, an amino acid linker sequence may be employed to separate a first and a second polypeptide by a distance sufficient to ensure that each polypeptide folds, if necessary, into its higher order structure (e.g., secondary, tertiary, quaternary structures). The amino acid linker sequence can be incorporated into the fusion polypeptide using standard techniques well known in the art.

If desired, suitable peptide linker sequences can be chosen based on such factors as their ability to adopt a flexible extended conformation; their inability to adopt a structure that could interfere with functional epitopes on the at least one antigen; and/or the lack of residues (e.g., hydrophobic or charged) that might react with any functional epitopes. Preferably, peptide linker sequences comprise Gly, Val, Thr, Ser, Pro, and/or Ala residues. The linker sequence can have any suitable number of amino acid residues. Preferably, the linker sequence is at least 1 amino acid residue in length, illustratively, about 1 to about 200, about 5 to about 180, about 10 to about 160, about 15 to about 150, about 20 to about 140, about 30 to about 100, about 40 to about 80, about 50 to about 60 amino acid residues in length.

Chemical linkers also can include carbohydrate linkers, lipid linkers, fatty acid linkers, polyether linkers, e.g., PEG, etc.

Methods of joining also can include ionic interactions, for example by expressing negative and positive tails and indirect binding through antibodies and streptavidin-biotin interactions. (See, e.g., Bioconjugate Techniques, supra).

In some embodiments, the conjugate is prepared by recombinant expression of a nucleic acid encoding the at least one antigen and/or at least one polypeptide. For example, the fusion product (i.e., conjugate) can be made by ligating the appropriate nucleic acid sequences encoding the desired amino acid sequences to each other by methods known in the art, in the proper coding frame, and expressing the product by methods known in the art.

In some embodiments, recombinant nucleic acids encoding the conjugate, optionally, can be modified to provide preferred codons which enhance translation of the nucleic acid in a selected organism (e.g., yeast, bacteria).

Expression systems for producing the conjugate are well know to those of ordinary skill in the art. For example, the polynucleotide that encodes the conjugate can be placed under the control of a promoter (e.g., constituitive, regulatable) that is functional in the desired host cell. A variety of promoters (e.g., prokaryotic (e.g., lac promoter), eukaryotic (e.g., SV40, papilloma virus, Epstein-Barr, CMV) are known, and can be used in the expression vectors of the invention, depending on the particular application. Preferably, the promoter selected depends upon the cell (e.g., yeast, bacteria, antigen-presenting cell, muscle cell, etc.) in which the promoter is to be active. Other expression control sequences such as ribosome binding sites, transcription termination sites, and the like also can, optionally, be included. Generally, prokaryotic and eukaryotic expression systems for bacteria, mammalian cells, yeast, and insect cells are well known in the art and are also commercially available.

If desired, the conjugate, recombinantly prepared or otherwise, can be purified according to standard procedures well known in the art, including ammonium sulfate precipitation, affinity columns, column chromatography, gel electrophoresis, and the like. Optionally, substantially pure preparations can be prepared, preferably preparations having at least about 90 to 95% homogeneity with respect to the conjugate. To facilitate purification, optionally, the conjugates also can include a coding sequence for an epitope or tag for which an affinity binding reagent is available, for example myc, polyhistidine, etc.

In other embodiments, the conjugate, optionally, can further comprise one or more immunomodulatory molecule, for example one or more cytokines (e.g., G-CSF, M-CSF, GM-CSF, IL-1, IL-2, IL-3G, IL-4, Il-6, Il-7, TNF, and the like). Thus, if desired, combinations of cytokines, which can provide an enhanced immune response such as a synergistic response as compared to the response produced by a single cytokine, can be utilized.

In another embodiment, the conjugate, optionally, can be coupled or bonded to a monoclonal antibody or a binding fragment thereof (e.g., Fv, scFv) specific for a particular surface structure of a target cell (e.g., an antigen-presenting cells, muscle cells), thereby providing for a concentration of the at least one antigen to the target cell. The antibody or the binding fragment thereof, therefore, effectively can act as a delivery vehicle for targeting antigenic determinants onto macrophage cells and B-cells, for example, thereby facilitating their recognition by the T-helper cells. The presenting cells can possess a variety of specific cell surface structures or markers, which can be targeted by a particular monoclonal antibody or binding fragment thereof. Thus, for example, the at least one antigen can be coupled to a monoclonal antibody or a binding fragment thereof specific for any of the surface structures on the antigen presenting cells, including Class I and Class II MHC gene products.

The surface structures on the antigen presenting cells of the immune system that can be recognized and targeted by the antibody portion of the conjugate are numerous and the specific such surface antigen structure targeted by the monoclonal antibody or binding fragment thereof can depend on the specific antibody. The monoclonal antibody or a binding fragment thereof can be provided specific for a gene product of the MHC, and, in particular, can be specific for class I molecules of MHC or for class II molecules of MHC. However, the invention is not limited to such specific surface structures and the conjugates comprising the corresponding monoclonal antibodies or binding fragments thereof but rather, as will be apparent to those skilled in the art, the invention is applicable to any other convenient surface structure of a target cell which can be recognized and targeted by a specific monoclonal antibody or binding fragment thereof (e.g., dendritic- and CD4 cell-specific monoclonal antibody or binding fragment thereof).

### The Double-Stranded Polynucleotides

In other aspects, the present invention provides an immunogenic composition comprising a conjugate comprising at least one antigen conjugated to at least one polypeptide capable of binding a double-stranded polynucleotide, wherein the immunogenic composition further comprises the double-stranded polynucleotide. The at least one antigen and the at least one polypeptide are as described above.

Generally, the double-stranded polynucleotide comprises a region having two or more nucleotides that are in a double-stranded conformation. Accordingly, the double-stranded polynucleotide can further comprise single-stranded regions. Furthermore, when formed from only one strand, a double-stranded polynucleotide can take the form of a self-complementary hairpin-type molecule that doubles back on itself to form a duplex. For example, double-stranded polynucleotide can be a single molecule with a region of self-complimentarily such that nucleotides in one segment of the molecule base pair with nucleotides in another segment of the molecule.

In one embodiment, the double-stranded polynucleotide is a single contiguous strand comprising ribonucleotides, deoxyribonucleotides, or a mixture of ribonucleotides and deoxyribonucleotides, such as, but not limited to, RNA/RNA, DNA/DNA, and RNA/DNA hybrids. In another embodiment, the single contiguous strand comprises a region of ribonucleotides that is hybridized to a region of ribonucleotides or a region of deoxyribonucleotides.

The double-stranded polynucleotide also can include two different strands that have a region of complimentarily to each other. In various embodiments, both strands comprise ribonucleotides, one strand comprises ribonucleotides and one strand comprises deoxyribonucleotides, or one or both strands comprise a mixture of ribonucleotides and deoxyribonucleotides.

Preferably, the double-stranded regions of the double-stranded polynucleotide are at least about 50, 60, 70, 80, 90, 95, 98, or 100% complimentary. Preferably, the region of the double-stranded polynucleotide that is present in a double-stranded conformation includes at least about 5, 6, 8, 10, 15, 20, 25, 30, 35, 40, 45, 50, 55, 60, 70, 80, 90, 100, 150, 200, 300, 400, 500, 1000, 2000 or greater base pairs of nucleotides. In some embodiments, the double-stranded polynucleotide does not contain any single stranded regions, such as single stranded ends. In other embodiments, the double-stranded polynucleotide has one or more single stranded regions or overhangs.

In other embodiments, the double-stranded polynucleotide is a single circular nucleic acid comprising a sense and an antisense region, or the double-stranded polynucleotide comprises a circular nucleic acid and either a second circular nucleic acid or a linear nucleic acid.

The double-stranded polynucleotide, optionally, can include modified nucleotides, caps, naturally or non-naturally occurring linkages, and the like. For example, in one embodiment, the double-stranded polynucleotide is dsRNA, wherein the dsRNA comprises one or more modified nucleotides in which the 2′ position in the sugar contains a halogen (such as flourine group) or contains an alkoxy group (such as a methoxy group) that can increase the half-life of the dsRNA in vitro or in vivo compared to the corresponding dsRNA in which the corresponding 2′ position contains a hydrogen or an hydroxyl group. In another embodiment, the dsRNA includes one or more linkages between adjacent nucleotides other than a naturally-occurring phosphodiester linkage. Examples of such linkages include phosphoramide, phosphorothioate, and phosphorodithioate linkages. In other embodiments, the dsRNA contains one or two capped strands or no capped strands.

Generally, the double-stranded polynucleotides can be prepared using in vitro or in vivo methods known to persons of ordinary skill in the art. For example, methods of providing dsRNA include in vitro synthesis (e.g., chemical synthesis and in vitro transcription) and in vivo transcription. For example, in vitro synthesis of dsRNA may be achieved by synthesizing sense and antisense RNA from DNA templates using T7 polymerase and subsequent hybridization to form dsRNA. Alternatively, transcription can be performed using an expression vector comprising promoters on opposite ends of a designated DNA sequence in which the promoters are oriented towards each other and capable of transcribing a strand of DNA to produce two resulting transcripts that can hybridize, thereby giving rise to a dsRNA molecule. Double-stranded DNA can be chemically synthesized or prepared from natural (e.g., genomic) or non-natural sources using one or more techniques known in the art including, but not limited to, PCR and enzymatic digestion.

In one embodiment, the double-stranded polynucleotide comprises poly(dA), poly(dC), poly(dG), poly(dI), poly(dT), poly(A), poly(C), poly(G), poly(I), or poly(U).

In another embodiment, the double-stranded polynucleotide comprises one or more regions of duplex DNA selected from poly(dI)/poly(dT), poly(dG)/poly(dC), poly(dI)/poly(dC), or poly(dA)/poly(dT).

In one embodiment, the double-stranded polynucleotide comprises one or more regions of duplex RNA comprising poly(I)/poly(C) or poly(G)/poly(C).

In another embodiment, the double-stranded polynucleotide comprises one or more regions of duplex DNA alternating copolymers of poly(dA-dT)/poly(dA-dT), poly(dI-dC)/poly(dI-dC), poly(dG-dC)/poly(dG-dC), or poly(dA-dC)/poly(dG-dT).

In other embodiments, the double-stranded polynucleotide comprises a Z-RNA or Z-DNA region.

Examples of double-stranded polynucleotides also are described in e.g., U.S. Publication Nos. 2007/0224219 and 2002/0142974; Brown et al., PNAS, 97:13532 (2000); Kwon et al., PNAS, 102:12759 (2005); Ichinoe et al., J. Virology, 79:2910 (2005); and Sugiyama et al., International Immunology, 20:1 (2007), each of which is incorporated herein by reference for its teaching of double-stranded polynucleotide.

### Ligands

Any ligand pair can be used in the present invention. One example includes the use of biotin and avidin. Other protein-protein ligand pairs maybe used. In addition, chemical ligands can also be used. A ligand is a molecule with an affinity to bind to a second atom or molecule. This affinity can be described in terms of noncovalent interactions, such as the type of binding that occurs in enzymes that are specific for certain substrates; or of a mode of binding where an atom or groups of atoms are covalently bound to a central atom, as in the case of coordination complexes and organometallic compounds. When a protein binds to another molecule, that molecule may be referred to as a ligand. The site where the ligand is bound is known as the binding or active site of the protein.

In certain embodiments, the ligand is made in a multimeric form to allow attachment of more than one adjuvant or TLR agonist. For example, when the dsRNA is biotinylated, a tetrameric avidin may be used. In this situation, one avidin binds the dsRNA and the other 3 avidins can be used to bind another dsRNA or other adjuvants or TLR agonists (or combinations thereof) that contain a biotin.

### III. COMPOSITIONS

In other aspects, the present invention provides an immunogenic composition comprising a conjugate comprising at least one antigen/small molecule wherein the at least one antigen is conjugated to at least one polypeptide capable of binding a double-stranded polynucleotide, wherein the immunogenic composition further comprises the double-stranded polynucleotide. The composition may also comprise ligand through which additional adjuvants can be complexed to the antigen/small molecule-polypeptide conjugate. The at least one antigen/small molecule, the at least one polypeptide, the double-stranded polynucleotide and ligands are as described herein.

In other embodiments, the compositions comprises an expression vector such as a viral vector. A polynucleotide encoding the antigen of interest is fused to a polynucleotide encoding the polypeptide that is capable of binding double stranded RNA (such as E3L). Suitable expression vectors are known and include, but are not limited to, a vaccinia virus vector, such as NYVAC or ALVAC II. The composition can be administered to a patient so that the patient expresses the antigen-polypeptide conjugate and in turn, the expressed conjugate elicits an immune response to the antigen within the patient. In other embodiments, the expression vector is administered to a non-human host to generate the conjugate, which in turn can then be isolated and purified and then administered to a patient.

In some embodiments, the composition can, optionally, comprise single stranded nucleic acid molecules that assume a double-stranded conformation under suitable conditions, or a combination of two single stranded nucleic acid molecules that are provided simultaneously or sequentially and that assume a double-stranded conformation under suitable conditions.

In another aspect, the present invention provides a composition comprising a conjugate as described above, or nucleic acids encoding the conjugate, wherein the composition further comprises a double-stranded polynucleotide as described above. In one embodiment, the composition is a pharmaceutically acceptable composition, wherein the composition further comprises a pharmaceutically acceptable carrier.

A pharmaceutically acceptable composition in accordance with the present invention, when administered to a subject, can elicit an immune response against the at least one antigen. The pharmaceutically acceptable compositions of the present invention can be useful as vaccine compositions for prophylactic or therapeutic treatment of a disorder or disease, or symptoms thereof.

In some embodiments, the pharmaceutically acceptable composition further comprises a physiologically acceptable carrier, diluent, or excipient.

Pharmaceutically acceptable carriers known in the art include, but are not limited to, sterile water, saline, glucose, dextrose, or buffered solutions. Agents such as diluents, stabilizers (e.g., sugars and amino acids), preservatives, wetting agents, emulsifying agents, pH buffering agents, additives that enhance viscosity, and the like. Preferably, the medium or carrier will produce minimal or no adverse effects.

In other embodiments, the pharmaceutically acceptable composition, optionally, further comprises one or more adjuvants or TLR agonists, in addition to the double-stranded polynucleotide. Any known suitable adjuvant or TLR agonists or combinations thereof may be used. Preferably, the one or more adjuvants or TLR agonists employed provide for increased immunogenicity. The one or more adjuvants TLR agonists may provide for slow release of antigen (e.g., the adjuvant can be a liposome), or it can be an adjuvant that is immunogenic in its own right thereby functioning synergistically with antigens. For example, the adjuvant can be a known adjuvant or other substance that promotes nucleic acid uptake, recruits immune system cells to the site of administration, or facilitates the immune activation of responding lymphoid cells. Adjuvants include, but are not limited to, immunomodulatory molecules (e.g., cytokines), oil and water emulsions, aluminum hydroxide, glucan, dextran sulfate, iron oxide, sodium alginate, Bacto-Adjuvant, synthetic polymers such as poly amino acids and co-polymers of amino acids, saponin, paraffin oil, and muramyl dipeptide. TLR agonists include, but are not limited to, TLR4 agonist, TLR 5 agonist, TLR9 agonist or TLR7/8 agonist.

### IV. METHODS

In another aspect, the present invention provides a method for eliciting in a subject an immune response to the at least one antigen or small molecule. The method comprises administering to the subject the pharmaceutically acceptable composition described above, wherein the composition, when administered to the subject, elicits the immune response to the at least one antigen/small molecule.

Generally, the immune response can include either the humoral or the cell-mediated immune response, or both. For example, antigen presentation through an immunological pathway involving MHC II proteins or direct B-cell stimulation can produce a humoral response; and, antigens presented through a pathway involving MHC I proteins can elicit the cellular arm of the immune system.

A humoral response can be determined by a standard immunoassay for antibody levels in a serum sample from the subject receiving the pharmaceutically acceptable composition. A cellular immune response is a response that involves T cells, and can be determined in vitro or in vivo. For example, a general cellular immune response can be determined as the T cell proliferative activity in cells (e.g., peripheral blood leukocytes (PBLs)) sampled from the subject at a suitable time following the administering of the pharmaceutically acceptable composition. Following incubation of e.g., peripheral blood mononuclear cells (PBMC) with a stimulator for an appropriate period, [³H]thymidine incorporation can be determined. The subset of T cells that is proliferating can be determined using flow cytometry. T cell cytotoxicity (CTh) can also be determined.

In one embodiment, the immune response that is elicited is sufficient for prophylactic or therapeutic treatment of a disease or disorder, or a symptom associated therewith. Accordingly, a beneficial effect of the pharmaceutically acceptable composition will generally at least in part be immune-mediated, although an immune response need not be positively demonstrated in order for the compositions and methods described herein to fall within the scope of the present invention.

Administering to both human and non-human vertebrates is contemplated within the scope of the present invention. Veterinary applications also are contemplated. Generally, the subject is any living organism in which an immune response can be elicited. Examples of subjects include, without limitation, humans, livestock, dogs, cats, mice, rats, and transgenic species thereof.

The pharmaceutically acceptable composition can be administered in a therapeutically or a prophylactically effective amount, either alone or in combination with one or more other antigens. Administering the pharmaceutically acceptable composition of the present invention to the subject can be carried out using known procedures, and at dosages and for periods of time sufficient to achieve a desired effect. For example, a therapeutically or prophylactically effective amount of the pharmaceutically acceptable composition, can vary according to factors such as the age, sex, and weight of the subject. Dosage regima can be adjusted by one of ordinary skill in the art to elicit the desired immune response including immune responses that provide therapeutic or prophylactic effects.

The route of administering can be parenteral, intramuscular, subcutaneous, intradermal, intraperitoneal, intranasal, intravenous (including via an indwelling catheter), via an afferent lymph vessel, or by any other route suitable in view of the neoplastic disease being treated and the subject's condition. Preferably, the dose will be administered in an amount and for a period of time effective in bringing about a desired response, be it eliciting the immune response, or the prophylactic or therapeutic treatment of the disease or disorder, or symptoms associated therewith.

The pharmaceutically acceptable composition can be given subsequent to, preceding, or contemporaneously with other therapies including therapies that also elicit an immune response in the subject. For example, the subject may previously or concurrently be treated by other forms of immunotherapy, such other therapies preferably provided in such a way so as not to interfere with the immunogenicity of the compositions of the present invention.

Administering can be properly timed by the care giver (e.g., physician, veterinarian), and can depend on the clinical condition of the subject, the objectives of administering, and/or other therapies also being contemplated or administered. In some embodiments, an initial dose can be administered, and the subject monitored for either an immunological or clinical response, preferably both. An immunological reaction also can be determined by a delayed inflammatory response at the site of administering. One or more doses subsequent to the initial dose can be given as appropriate, typically on a monthly, semimonthly, or preferably a weekly basis, until the desired effect is achieved. Thereafter, additional booster or maintenance doses can be given as required, particularly when the immunological or clinical benefit appears to subside.

As further illustrated below, a composition in accordance with the present invention can comprise the conjugate, or nucleic acids encoding the conjugate.

### Nucleic Acids

Generally, the subject can be inoculated with a pharmaceutically acceptable composition comprising nucleic acids through any parenteral route. For example, the subject can be inoculated by intravenous, intraperitoneal, intradermal, subcutaneous, inhalation, or intramuscular routes, or by particle bombardment using a gene gun. Preferably, muscle tissue can be a site for the delivery and expression of polynucleotides. A dose of polynucleotides can be administered into muscle by multiple and/or repetitive injections, for example, to extend administration over long periods of time. Thus, muscle cells can be injected with a composition comprising the double-stranded polynucleotide and polynucleotides coding for the conjugate, whereby the expressed conjugate can be presented by muscle cells in the context of antigens of the major histocompatibility complex to elicit the immune response against the at least one antigen.

The epidermis can be another useful site for the delivery and expression of polynucleotides, for example either by direct injection or particle bombardment. A dose of polynucleotides can be administered in the epidermis, for example by multiple injections or bombardments to extend administering over long periods of time. For example, skin cells can be injected. A subject also can be inoculated by a mucosal route. The polynucleotides can be administered to a mucosal surface by a variety of methods including polynucleotide-containing nose-drops, inhalants, suppositories, microsphere-encapsulated polynucleotides, or by bombardment with polynucleotide-coated gold particles.

Any appropriate physiologically compatible medium, such as saline for injection, or gold particles for particle bombardment, is suitable for introducing polynucleotides into a subject.

### RNA

In some embodiments, a pharmaceutically acceptable composition comprises nucleic acids encoding the polypeptides of the conjugate. In one embodiment, the encoding nucleic acids are RNAs. The RNAs comprise translatable RNA templates to guide the intracellular synthesis of amino acid chains that provide the conjugate. RNAs encoding the conjugate also can be in vitro transcribed, e.g., reverse transcribed to produce cDNAs that can then be amplified by PCR, if desired, and subsequently transcribed in vitro, with or without cloning the cDNA.

In another embodiment, the nucleic acids encoding the polypeptides of the conjugate comprise DNAs (e.g., a cDNA, expression vector, etc.) having open reading frames encoding the polypeptides of the conjugate. For example, a pharmaceutically acceptable composition comprising expression vectors having DNA open reading frames encoding the polypeptides of the conjugate can be administered to a subject.

When taken up by a cell (e.g., muscle cell, APC such as a dendritic cell, macrophage, etc.), a DNA molecule can be present in the cell as an extrachromosomal molecule and/or can integrate into the chromosome. DNA can be introduced into cells in the form of a plasmid which can remain as separate genetic material. Alternatively, linear DNAs that can integrate into the chromosome can be introduced into the cell. Optionally, when introducing DNA into a cell, reagents which promote DNA integration into chromosomes can be added.

Thus, in some embodiments, the DNAs in accordance with the present invention include regulatory elements necessary for expression of an open reading frame. Such elements can include, for example, a promoter, an initiation codon, a stop codon, and a polyadenylation signal. In addition, enhancers can be included. As is known in the art, these elements are preferably operably linked to a sequence that encodes the polypeptides corresponding to the conjugate. Regulatory elements are preferably selected that are operable in the species of the subject to which they are to be administered. Initiation codons and stop codons in frame with a coding sequence are preferably included.

Examples of promoters include but are not limited to promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, Cytomegalovirus (CMV) such as the CMV immediate early promoter, Epstein Barr Virus (EBV), Rous Sarcoma Virus (RSV) as well as promoters from human genes such as human actin, human myosin, human hemoglobin, human muscle creatine, and human metalothionein. Examples of suitable polyadenylation signals include but are not limited to SV40 polyadenylation signals and LTR polyadenylation signals.

In addition to the regulatory elements required for DNA expression, other elements may also be included in the DNA molecule. Such additional elements include enhancers. Enhancers include the promoters described hereinabove. Preferred enhancers/promoters include, for example, human actin, human myosin, human hemoglobin, human muscle creatine and viral enhancers such as those from CMV, RSV and EBV.

Optionally, the DNAs can be operably incorporated in a carrier or delivery vector. A variety of suitable delivery vectors are known in the art including, but not limited to, biodegradable microcapsules, immuno-stimulating complexes (ISCOMs) or liposomes, and genetically engineered attenuated live carriers such as viruses or bacteria.

Optionally, the DNAs also can be provided with reagents that improve the uptake of the genetic material by cells. For example, the DNA can be formulated with or administered in conjunction with an uptake facilitator reagent selected from the group consisting of benzoic acid esters, anilides, amidines, and urethans.

In various other aspects, the conjugate or nucleic acids encoding the conjugate also can provide for compositions and methods for providing antigen-primed antigen-presenting cells, and antigen-specific T lymphocytes generated with these antigen-presenting cells, for use as active compounds in immunomodulating compositions and methods for prophylactic or therapeutic applications.

Accordingly, in another aspect, the invention provides a method for making antigen-primed antigen-presenting cells, the method comprising: contacting antigen-presenting cells with an immunogenic composition comprising a double-stranded polynucleotide and a conjugate, or nucleic acids encoding the conjugate, in vitro under a condition sufficient for the at least one antigen to be presented by the antigen-presenting cells. The conjugate and the double-stranded polynucleotides are as described above.

The immunogenic composition can be contacted with a homogenous, substantially homogenous, or a heterogeneous cellular composition comprising antigen-presenting cells. For example, the cellular composition can include but is not limited to whole blood, fresh blood, or fractions thereof such as, but not limited to, peripheral blood mononuclear cells, buffy coat fractions of whole blood, packed red cells, irradiated blood, dendritic cells, monocytes, macrophages, neutrophils, lymphocytes, natural killer cells, and natural killer T cells. If, optionally, precursors of antigen-presenting cells are used, the precursors can be cultured under suitable culture conditions sufficient to differentiate the precursors into antigen-presenting cells. Preferably, the antigen-presenting cells (or, optionally, precursors) are selected from monocytes, macrophages, cells of myeloid lineage, B cells, dendritic cells, or Langerhans cells.

The amount of the immunogenic composition to be placed in contact with antigen-presenting cells can be determined by one of ordinary skill in the art by routine experimentation. Generally, antigen-presenting cells are contacted with the immunogenic composition for a period of time sufficient for cells to present the processed forms of the at least one antigen for the modulation of T cells. In one embodiment, antigen-presenting cells are incubated with the immunogenic composition for less than about a week, illustratively, for about 1 minute to about 48 hours, about 2 minutes to about 36 hours, about 3 minutes to about 24 hours, about 4 minutes to about 12 hours, about 6 minutes to about 8 hours, about 8 minutes to about 6 hours, about 10 minutes to about 5 hours, about 15 minutes to about 4 hours, about 20 minutes to about 3 hours, about 30 minutes to about 2 hours, and about 40 minutes to about 1 hour. The time and amount necessary for the antigen presenting cells to process and present the antigens can be determined, for example using pulse-chase methods wherein contact is followed by a washout period and exposure to a read-out system e.g., antigen reactive T cells.

Typically, the length of time necessary for an antigen-presenting cell to present an antigen on its surface can vary depending on a number of factors including the antigen or form (e.g., peptide versus encoding polynucleotide) of antigen employed, its dose, and the antigen-presenting cell employed, as well as the conditions under which antigen loading is undertaken. These parameters can be determined by the skilled artisan using routine procedures. Efficiency of priming of an antigen-presenting cell can be determined by assaying T cell cytotoxic activity in vitro or using antigen-presenting cells as targets of CTLs. Other methods that can detect the presence of antigen on the surface of antigen-presenting cells are also contemplated by the presented invention.

A number of methods for delivery of antigens to the endogenous processing pathway of antigen-presenting cells are known. Such methods include but are not limited to methods involving pH-sensitive liposomes, apoptotic cell delivery, pulsing cells onto dendritic cells, delivering recombinant chimeric virus-like particles (VLPs) comprising antigen to the MHC class I processing pathway of a dendritic cell line.

In one embodiment, the conjugate and the double-stranded polynucleotide are incubated with antigen-presenting cells. In other embodiments, the conjugate and the double-stranded polynucleotide can be coupled to a cytolysin to enhance the transfer of the at least one antigen into the cytosol of an antigen-presenting cell for delivery to the MHC class I pathway. Exemplary cytolysins include saponin compounds such as saponin-containing Immune Stimulating Complexes (ISCOMs), pore-forming toxins (e.g., an alpha-toxin), and natural cytolysins of gram-positive bacteria such as listeriolysin O (LLO), streptolysin O (SLO), and perfringolysin O (PFO).

By way of another example, in other embodiments, antigen-presenting cells, preferably dendritic cells and macrophage, can be isolated according to methods known in the art and transfected with polynucleotides by methods known in the art for introducing double-stranded polynucleotides and nucleic acids encoding the conjugate into the APCs. Transfection reagents and methods (e.g., SuperFect®) also are commercially available. For example, the polynucleotides and nucleic acids encoding the conjugate can be provided in a suitable medium (e.g., Opti-MEM®) and combined with a lipid (e.g., a cationic lipid) prior to contact with APCs. Non-limiting examples of lipids include LIPOFECTIN™, LIPOFECTAMINE™, DODAC/DOPE, and CHOL/DOPE. The resulting polynucleotide-lipid complex can then be contacted with APCs. Alternatively, the polynucleotide can be introduced into ACPs using techniques such as electroporation or calcium phosphate transfection. The polynucleotide-loaded APCs can then be used to stimulate cytotoxic T lymphocyte (CTL) proliferation in vivo or ex vivo. In one embodiment, the ex vivo expanded CTL is administered to the subject in a method of adoptive immunotherapy. The ability of the polynucleotide-loaded antigen-presenting cells to stimulate a CTL response can be determined by known methods, for example by assaying the ability of effector cells to lyse a target cell. Methods and compositions using antigen-presenting cells loaded with e.g., RNA are described in U.S. Pat. No. 6,306,388 to Nair et al., which is incorporated herein by reference in its entirety.

In another aspect, the present invention provides a composition comprising antigen-presenting cells that have been contacted in vitro with the conjugate and the double-stranded polynucleotides described above, under a condition sufficient for the at least one antigen to be presented by the antigen-presenting cells.

In another aspect, the present invention provides a method for preparing lymphocytes specific for the at least one antigen. The method comprises contacting lymphocytes with the antigen-presenting cells described above under conditions sufficient to produce at least one antigen-specific lymphocyte capable of eliciting an immune response against the at least one antigen. Thus, the antigen-presenting cells also can be used to provide lymphocytes, including T lymphocytes and B lymphocytes, for eliciting an immune response against the at least one antigen.

In one embodiment, a preparation of T lymphocytes is contacted with the antigen-presenting cells described above for a period of time, preferably for at least about 24 hours, for priming the T lymphocytes to the at least one antigen presented by the antigen-presenting cells.

T lymphocytes can be obtained from any suitable source such as peripheral blood, spleen, and lymph nodes. The T lymphocytes can be used as crude preparations or as partially purified or substantially purified preparations, which can be obtained by standard techniques including but not limited to methods involving immunomagnetic or flow cytometry techniques using antibodies.

In another aspect, the present invention provides a method for eliciting an immune response to the at least one antigen, the method comprising administering to the subject the antigen-presenting cells or the lymphocytes described above in effective amounts sufficient to elicit the immune response. In one embodiment, the antigen-presenting cells or the lymphocytes are administered systemically, preferably by injection. Alternately, one can administer locally rather than systemically, for example, via injection directly into tissue, preferably in a depot or sustained release formulation. Furthermore, one can administer in a targeted drug delivery system, for example, in a liposome that is coated with tissue-specific antibody. The liposomes can be targeted to and taken up selectively by the tissue. In another embodiment, the invention provides use of the antigen-presenting cells or the lymphocytes in the preparation of a medicament for eliciting an immune response to the at least one antigen.

Accordingly, the antigen-primed antigen-presenting cells of the present invention and the antigen-specific T lymphocytes generated with these antigen-presenting cells can be used as active compounds in immunomodulating compositions for prophylactic or therapeutic applications. In some embodiments, the antigen-primed antigen-presenting cells of the invention can be used for generating CD8+ or CD4+ CTL, for adoptive transfer to the subject.

Techniques for formulating and administering can be found in Remington's Pharmaceutical Sciences, Mack Publishing Co., Easton, Pa., latest edition. Suitable routes can, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. For injection, the therapeutic/prophylactic compositions of the present invention can be formulated in aqueous solutions, preferably in physiologically compatible buffers such as Hanks' solution, Ringer's solution, or physiological saline buffer.

### IV. ANTIBODIES

The compositions of the present invention also can be used as immunogens to provide antibodies against the at least one antigen. Accordingly, in other aspects, the composition and methods of the present invention provide antibodies against the at least one antigen, which antibodies themselves have many uses, for example in methods for passive immunization or for diagnostic tests and kits based upon immunological binding.

### V. KITS

The compositions of the present invention can be supplied in unit dosage or kit form. Kits can comprise various components of the conjugate, the ligand, the double stranded nucleic acid, additional adjuvants and/or TLR agonists, pharmaceutically acceptable composition or vaccines thereof provided in separate containers. For example, the containers can separately comprise the polypeptide(s) of the conjugate, or nucleic acids encoding the polypeptide(s) of the conjugate such that when combined with other components of the kit together constitute a pharmaceutically acceptable composition in unit dosage or multiple dosage form. Preferred kits at least comprise, in separate containers, the polypeptide(s) of the conjugate or nucleic acids encoding the conjugate; and the double-stranded polynucleotide. The kit can further comprise a physiologically acceptable carrier, diluent, or excipient in a separate container. Optionally, the kit can further comprise a delivery agent such as nanoparticles or transfection reagents. Packaged compositions and kits of this invention also can include instructions for storage, preparation, and administering.

The present invention will be illustrated in more detail by way of Examples, but it is to be noted that the invention is not limited to the Examples.

### EXAMPLES

### Example 1

### Presentation of an Antigen Bound to dsRNA

To determine if presentation of an antigen bound to dsRNA increases immunogenicity of that antigen, a non-cleavable form of HIV gp160 is fused to the N-terminus of E3L and expressed in vaccinia virus. As a control, antigen is expressed fused to an E3L protein that does not bind to dsRNA (there are numerous mutants of E3L available for analysis). Mice are vaccinated by scarification and blood and splenocytes are harvested. Antibody levels to gp160 are determined by ELISA, and ELISpot assays and ICS are used to quantify the cellular immune response to gp160, in particular determining the breadth and quality of the response to gp160. Efficacy is defined as either increased humoral or cell mediated immunity after fusion to an E3L protein that binds dsRNA.

Immunogenicity of soluble gp160 fused to E3L protein is also investigated. His-tagged fusion protein is made in CHO cells to ensure proper glycosylation of gp160. Protein is denatured and bound to Ni+2 purification resin to remove any cellular RNA bound to E3L. Protein is re-natured on the resin and loaded with synthetic dsRNA. Unbound dsRNA is removed by washing. Protein/dsRNA complexes are eluted by incubation with imidazole and used for immunization of mice (either IM or IP). Again, fusion proteins containing E3L proteins that do not bind to dsRNA are used as a control. Immunogenicity is analyzed as described above.

To determine if multiple adjuvants or aggregation can increase immunogenicity of gp160, dsRNA bound fusion protein complexes are made as described above, expect that poly-biotinylated dsRNA is used. Complex on the column is incubated with varying concentrations of tetrameric avidin, followed by incubation with biotinylated LPS (commercially available) and biotinylated CpG DNA (available as custom synthesized DNS). Complexes are eluted, and aggregate size is determined by gel filtration. Animals are immunized and immune responses are analyzed as described above.

### Example 2

### Synthesis of an Imiquimod Analogue Tethered to Biotin

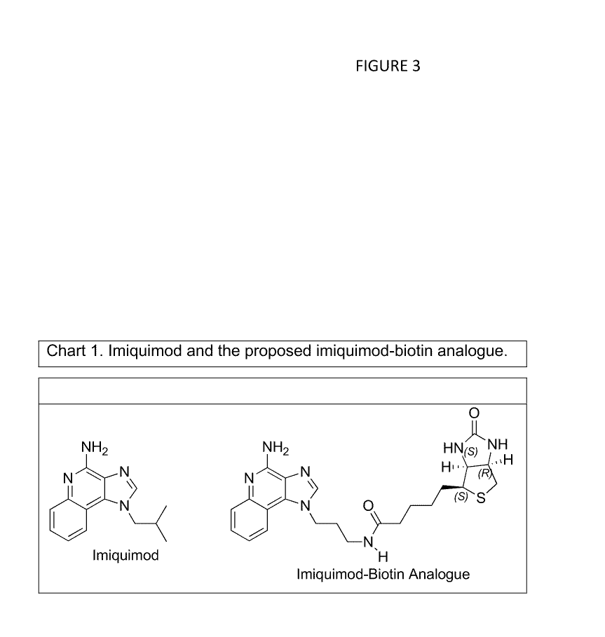

Imiquimod is a potent inducer of interferon (IFN) that has utility in treating skin diseases such as external genital warts. (8) The synthesis of the imiquimod-biotin analogue is shown in Chart 1 (FIG. 3). The present invention contemplates complexing this analogue with avidin and thereby induce IFN and ultimately antibody formation. It should be noted that derivitization of imiquimod at the 2 position of the heterocyclic ring structure had very little effect on activity of the compounds tested (7), suggesting that derivitization with biotin at this position should not compromise activity.

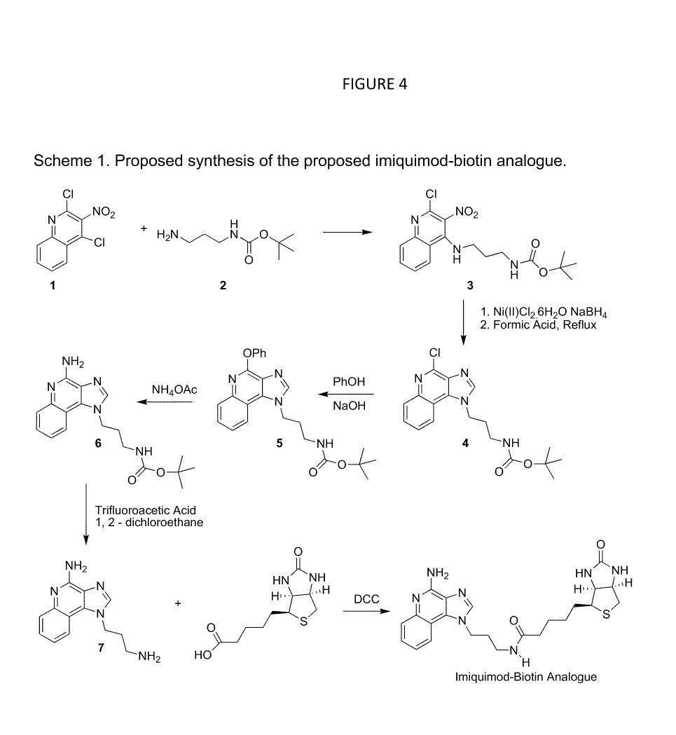

Imiquimod is a derivative of the 1H-imidazo[4,5-c]quinoline heterocyclic ring system. Although there are reports of the synthesis of this ring system (7, 14) in the literature, there are no reports of the synthesis of imiquimod tethered to biotin. An exemplary synthesis of this analogue is outlined in Scheme 1 (FIG. 4).

The synthesis outlined in Scheme 1 is based on a reported 1H-imidazo[4,5-c]quinoline synthesis. (10) The tether will be added utilizing a nucleophilic aromatic substitution reaction between 2,4-dichloro-3-nitroquinoline 1 (10) and amine 2 (27). The resulting product 3 will then be cyclized to the 1H-imidazo[4,5-c]quinoline ring system 4 by borohydride-mediated nitro group reduction followed by Phillips ring (24) closure with formic acid. A two-step process previously described in the literature (10) will introduce the amino group to afford 6. The coupling of the imiquimod derivative to biotin will involve the removal of the t-BOC blocking group from 6 with trifluoroacetic acid to afford 7 followed by dicyclohexylcarbodimide (DCC)-mediated coupling of 7 with biotin.

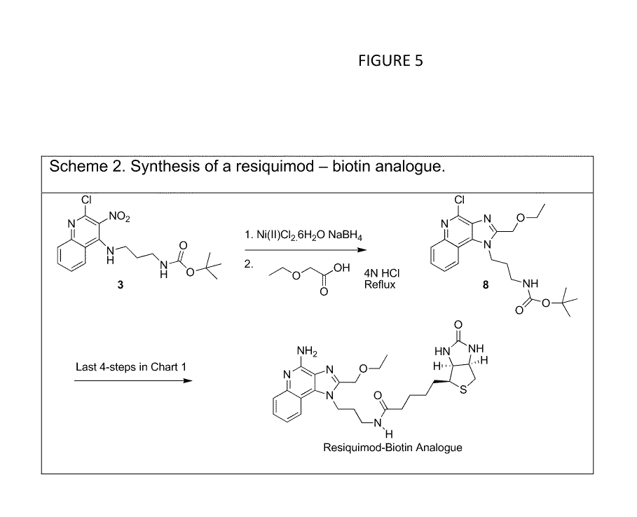

In order to optimize IFN induction the potent 1H-imidazo[4,5-c]quinoline analogue, resiquimod, is tethered to biotin (Scheme 2) (FIG. 5). Furthermore, the role of tether length on IFN induction will be investigated. The three-carbon chain tether utilized above (Scheme 1) will be extended to six carbons or more.

### Example 3

### Preparation of the Resiquimod-Biotin Analogue

Scheme 2 shows the preparation of the resiquimod-biotin analogue starting with intermediate 3 shown in Scheme 1. Reduction of 3 followed by Phillips (24) ring closure with ethoxyacetic acid will afford 8. Subjecting 8 to the last four steps shown in Scheme 1 will provide the tethered resiquimod analogue.

### Example 4

### Preparation of an Analogue Bearing a Six-Carbon 1H-Imidazo[4,5-c]Quinoline to Biotin Tether

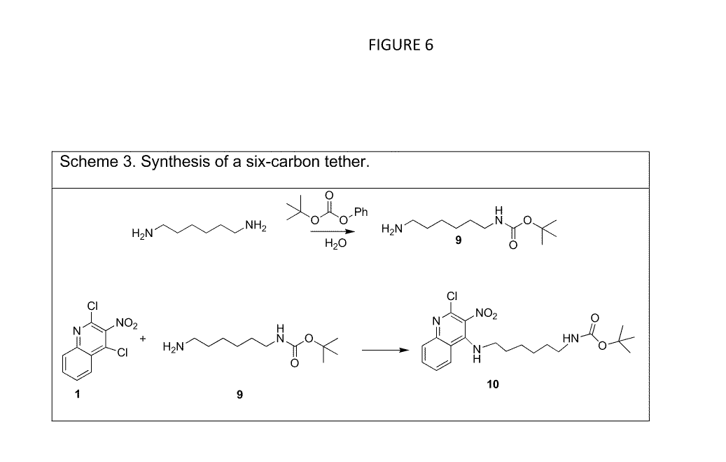

Scheme 3 (FIG. 6) shows the preparation of an analogue bearing a six-carbon 1H-imidazo[4,5-c]quinoline to biotin tether. Monoacylation of 1,6-hexanimine (commercially available) will be carried out utilizing phenyl t-BOC in the presence of water (27) to afford 9. The nucleophilic aromatic substitution reaction between 9 and 1 (Scheme 1) will afford 10 that could be converted to either a imiquimod or a resiquimod derivative tethered to biotin (see Schemes 1 and 2).

### Example 5

### Cocaine Haptens

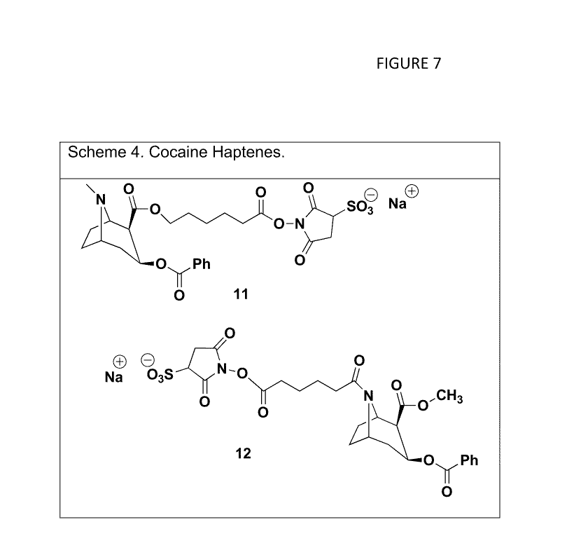

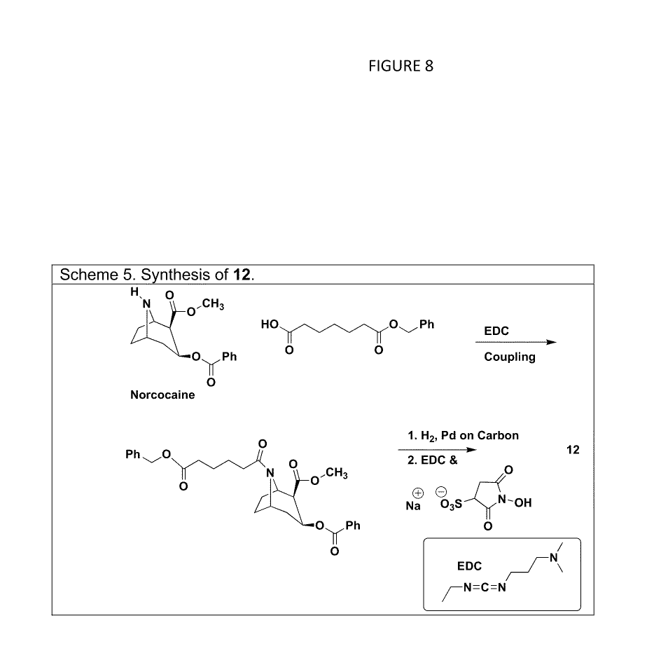

The cocaine haptens 11 and 12 shown in Scheme 4 (FIG. 7) are used to form cocaine immunoconjugates. The preparation of 11 has been reported in the literature. (1) Hapten 12 has not been reported and its proposed synthesis from norcocaine is outlined in Scheme 5 (FIG. 8). The synthetic methodology shown in Scheme 5 has precedents in the literature. (1)

### Example 6

### Synthesis, Purification and Haptenization of Proteins

A GST-E3L fusion protein is expressed in and purified from E. coli. Purified protein is assayed for the presence of endotoxin, as per manufacturers recommendation (Clonegen) and further purified, as necessary to remove any contaminating endotoxin (18). Fusion protein is bound to low molecular weight biotinylated dsRNA (to protect lysine residues necessary for binding to dsRNA) and haptenized with succinyl-norcocaine, as previously described (17, 19) (GST has 14 available lysine groups for haptenization and E3L has 9 lysine residues not involved in dsRNA-binding that are available for haptenization). Haptenated protein/dsRNA complexes are captured by binding to avidin-agarose, and protein is eluted from the dsRNA in high salt. A mutant of E3L that does not bind dsRNA, fused to GST is used as a control. Level of haptenization is determined either by UV absorbence, or by titering the number of free amino groups with TNBS, before and after haptenization (17).

5′-biotinylated dsRNA is synthesized in vitro. A 150 base pair cassette with a single A residue at each end of the cassette (and no internal A residues) is synthesized and cloned in both orientations into pBluescript. Plasmid is linearized downstream of the cassette, and RNA with a single biotinylated U residue is transcribed according to the manufacturers recommendations (Epicentre Biotechnologies), using biotin-16-UTP as a substrate. Complimentary RNAs are hybridized to one another, and excess ssRNA is digested with RNaseA. If dsRNA with increased amounts of biotin is necessary to obtain optimal sized particles, the cassette is increased in size in increments of 16 base pairs (1.5 turns of a dsRNA helix, putting biotin on opposite sides of the helix), with an additional A residue at the beginning of the cassette.

### Example 7

### Norcocaine-GST-E3L-dsRNA Particle Formation

Biotinylated dsRNA is incubated with various molar ratios of Norcocaine-GST-E3L, ranging from a 1:1 molar ratio (average molecule of dsRNA is loaded with 1 molecule of protein, 37% of the molecules of dsRNA will have no protein bound) to a protein/dsRNA molar ratio of 5 (average molecule of dsRNA loaded with 5 molecules of protein, 1% of the molecules of dsRNA will have no protein bound). Animals are vaccinated with protein bound to dsRNA, and immunogenicity is determined (as noted above), compared to Norcocaine-GST-E3L not loaded with dsRNA and compared to haptenated KLH and CTB. It is expected that conjugate bound to dsRNA will induce a higher titer of anti-cocaine antibodies, of higher avidity, than conjugates not bound to dsRNA (Norcocaine-GST-E3L, Norcocaine-KLH and Norcocaine-CTB).

### Example 8

### Formation of Particles Differing in Size and Degree of Cross-Linking

In order to determine if varying the particle size can influence immunogenicity, biotinylated dsRNA is incubated with varying molar ratios of avidin to crosslink the dsRNA. Particle size is determined by gel filtration (a Superose 6 FPLC column that can resolve particles up to 4×106 MW is used). Initially 10:1, 3:1, 1:1, 1:3 and 1:10 biotin to monomeric avidin molar ratios is tested, with the goal of obtaining monomeric dsRNA and particles of varying average sizes. These particles are loaded with the optimal ratio of purified, haptenated GST-E3L, as defined in the previous paragraph, and complexes are purified by gel filtration chromatography. Animals are immunized with different average sized particles, and immunogenicity is compared to haptenized GST-E3L not loaded with dsRNA, and compared to haptenized KLH and CTB. Particle sizes giving the best immunogenicity are further analyzed.

### Example 9

### Effects of Multiple Adjuvants on Immunogenicity

To determine if multiple adjuvants can increase immunogenicity of cocaine haptenated E3L, the optimal sized dsRNA protein complexes are further loaded with biotinylated adjuvants (LPS, CpG DNA, flagellin or TLR 7/8 agonists). Complexes are purified from free adjuvants by gel filtration chromatography. Particles are initially loaded with one of the adjuvant molecules and tested for immunogenicity. For any adjuvant molecules that increase immunogenicity of the dsRNA-protein complexes, combinations of molecules (e.g., biotinylated LPS and biotinylated CpG DNA) are tested. These experiments should allow one to determine the optimal particle size and optimal combination of adjuvant molecules to obtain the highest titer and highest affinity of anti-cocaine antibodies.

If endotoxin present in protein prepared form E. coli turns out to be an insurmountable problem, protein will be purified from baculovirus-infected insect cells. If high levels of haptenization are not obtained with the GST-E3L fusion protein, a tail with a high lysine content can be added to the fusion protein.

### Example 10

### Vaccine Construction

Functionalized cocaine with different chemical linkers is conjugated to modified oligonucleotides, which can then be assembled onto DNA-nanoscaffolds through Watson-Crick base-paring between the modified oligonucleotides and the single-stranded DNA attached on the DNA-nanoscaffolds. The number, position and neighboring distance of cocaine epitopes can be readily controlled using the self-assembling DNA-nanostructure. To induce T-cell dependent humoral responses, a protein, strepavidin, is added onto the DNA-nanoscaffolds, through its interaction with bionitylated oligonucleotides. Finally, CpG DNA (a TLR 9 agonist), and dsRNA (a TLR 3 agonist), can also be engineered directly onto the DNA-nanoscaffolds, in which an extended sequence of the CpG-DNA or dsRNA will be designed complementary to the anchoring sequence on the DNA-nanoscaffolds, and therefore will allow the controllable addition of the multiple adjuvants to the particle.

Taken together, tunable DNA nanoscaffold platform of the present invention is utilized to assemble several components needed for a robust cocaine vaccine, i.e., cocaine epitopes, T cell antigen-containing proteins and multiple adjuvants. The robust and versatile nature of various components that can be assembled onto the scaffolds, as well as the precision control over the number, position and configuration of assembling molecules in the multiplex design, makes the DNA nanotechnology an optimal platform on which to create multi-functional molecules.

### Example 11

### Immunological Assays

To determine the immunogenicity of constructed vaccines, 6-8 wks old Balb/C mice are immunized with the vaccines described above, and control vaccines, and the level of anti-cocaine antibodies are determined. Specifically, groups of 6 Balb/c mice are immunized subcutaneously with 100 μg of conjugate protein, with particles containing 100 μg of conjugate protein, or with DNA nanoparticle containing the equivalent amount of norcocaine, as previously described (12). Animals are immunized for 4 weeks, at one week intervals, with bleeds taken one week after the second and fourth immunizations. Antibody titers are determined by end-point ELISA, using norcocaine-derivatized BSA as the capture antigen. For potent immunogens, a titer of >25,000 is expected (2). Average antibody avidity is measured by equilibrium dialysis with [3H]cocaine, as previously described (20). For potent immunogens a low nM average avidity for cocaine is expected (20).

### Example 12

### Behavioral Experiments

### Overview.

The evaluation of the vaccines' effects on behavior is carried out by Co-investigator, Dr. Janet Neisewander, in Psychology at Arizona State University. Behavioral analyses will initially evaluate time-dependent effects of the vaccines on cocaine-induced locomotor activity. These tests allow screening for overt behavioral effects of the vaccines when administered alone or in combination with cocaine and whether positive effects of the vaccines persist over the course of 3 months. The most promising vaccines that most effectively reduce or eliminate cocaine-induced locomotor activity over a long duration are tested for their ability to reduce the reinforcing effects of cocaine self-administration. The effects of the vaccines on cocaine self-administration provide an initial screen for potential therapeutic efficacy toward reducing cocaine intake.

### Effects of Vaccines on Spontaneous and Cocaine-Induced Activity.

Procedure: Male Sprague-Dawley rats weighing 250+25 g at the start of the experiment are housed individually in a temperature controlled colony room with 12 hr light:dark cycle. The rats are handled for 1 week prior to the beginning of each experiment. Locomotor activity and stereotypy is video-taped while the rats are in a Plexiglas cage (44×24×20 cm high) that has a metal bar floor suspended over bedding and a thin metal bar ceiling. The behaviors are tracked using Clever Systems software that provides measures of rearing, small head movements, and distance traveled. Repetitive rearing and head movements are components of cocaine-induced stereotypic behaviors. First, baseline (pre-vaccine) measures of spontaneous and cocaine-induced activity are obtained. The rats are placed into the test cages for a 1-h habituation period, by the end of which most animals exhibit little activity. Rats are then administered 15 mg/kg, IP, cocaine-HCl and returned to the test cage for another 1-h period. Rats are assigned to 1 of 2 groups, counterbalanced for level of activity from the baseline test, that will receive either vaccine or the blank nano-particle platform as a control (n=8/condition). Beginning one week after administration of the vaccine and repeating 3 times once every 4 weeks thereafter, rats are tested for spontaneous activity (i.e., during habituation) and cocaine-induced activity as described above.

### Statistical Analyses and Predicted Outcomes.

Distance traveled, small head movements, and rearing are analyzed using mixed factor ANOVAs with test days (baseline and 4 post-vaccine tests) as a repeated measure and vaccine condition as a between subjects measure. Interactions are further analyzed using Newman-Keuls pairwise comparisons. In controls spontaneous activity will either not change or may decrease slightly across repeated tests, whereas cocaine-induced activity will become sensitized across repeated tests. Vaccines will not have an effect on spontaneous activity, but will reduce or eliminate cocaine-induced behaviors and that this effect will persist, at least to some extent, throughout the 3 months of testing.

### Effects of Vaccines on Cocaine Self-Administration.

Surgery: Intravenous cocaine infusions are delivered via surgically implanted jugular catheters with headmounts as described by Neisewander et al. (21) and detailed in the vertebrate animals section. Catheters are flushed daily with 0.1 ml solution of saline, heparin (50 U/ml) and ticarcillin (200 mg/ml) to maintain patency. Based on past experience, it is expected that catheter patency will be successfully maintained throughout the experiment in approximately 85% of the animals.

### Cocaine Self-Administration Training and Testing.

After at least 5 days of recovery from surgery, training sessions begin and occur for 2 h, 6 days/week at the same time each day. Sessions take place in operant conditioning chambers equipped with two levers mounted on the front wall, a cue light above one lever, a tone generator (500 Hz, 10 db above background), and a house light mounted on the center of the back wall. Rats are trained to press a lever reinforced by cocaine infusions (0.75 mg/kg/0.1 ml, IV) beginning on a fixed ratio (FR) 1 schedule and progressing to a FR 5 schedule. Initially, rats are food-restricted to 18 g, which facilitates acquisition of drug self-administration (3). After rats have achieved a criterion of 7 reinforces/h on the FR 5 schedule, food rations are gradually increased to ad libitum access over the next 3 days and thereafter animals have free access to food in their home cages. The lever with the cue light above is designated as the active lever and the other as the inactive lever. Schedule completions on the active lever will simultaneously activate the cue light, house light, and tone, followed one second later by a cocaine infusion (0.75 mg/kg/0.1 ml, IV). Upon completion of the 6-s infusion, the cue light, tone, and infusion pump is inactivated. The house light remains on for a 20-s timeout period during which lever presses have no scheduled consequences. Responses on the inactive lever will be recorded but will have no scheduled consequences.

After rats have reached a stability criterion of less than 10% variability in cocaine infusions obtained across 3 consecutive days with no upward or downward trends, a within session cocaine dose-effect function will be generated. Rats will have access to one of 5 doses of cocaine, with each dose available successively for a 30-min period in ascending order (0, 0.032, 0.10, 0.32, and 1.0 mg/kg/0.1 ml, IV), with a 5-min timeout period between each dose. Each 30-min test period begins with an experimenter-delivered infusion in order to clear the catheter of the previous dose. If animals fail to respond within 3 min, they will receive another infusion. This procedure is repeated 2-3 times, with at least 2 maintenance sessions intervening (i.e., 2-hr session with training dose), in order to establish a stable within session dose-effect function. Rats are then assigned to 1 of 2 groups, counterbalanced for level previous cocaine intake, that will receive either vaccine or the blank nano-particle platform as a control (n=12/condition). Beginning one week after administration of the vaccine and repeating 3 times once every 4 weeks thereafter, rats are tested for cocaine self-administration using the within session dose-effect function procedure. An identical procedure was used to generate the preliminary data (see Examples 13 and 14) except that animals were only tested once after receiving viral vector because of the transient nature of this manipulation.

### Statistical Analyses and Predicted Outcomes.

Infusions/30 min are analyzed using mixed factor ANOVAs with dose of cocaine as a within subjects measure and vaccine condition as a between subjects variable. Interactions are further analyzed using tests of simple main effects and pair-wise Newman-Keuls tests. Control rats should exhibit an inverted U-shaped dose-effect function that will be stable across repeated tests. Vaccinated rats should exhibit a flattened, downward shift in the dose-effect function that will become more dramatic across tests, indicative of marked attenuation, or perhaps even elimination, of cocaine reinforcing effects.

### Example 13

### Expression, Purification and Characterization of GST-E3L Protein

GST-E3L protein has been successfully expressed and purified from pGEX in E. coli. pGEX-6P-1, pGEXE3L and pGEX expressing various mutants of E3L were transformed into BL21(DE3) pLysS bacteria (Invitrogen) as per manufacturer's instructions. Cultures were grown to an O.D. 600 of 0.700 and then transgene synthesis was induced by the addition of IPTG for 2 hours at 30° C. Following induction, the cultures were harvested and lysed by sonication. Following sonication, NP-40 detergent was added to the cell extracts and the extracts were then rocked for 30 min at 4° C. Cell debris was removed by centrifugation. Washed Glutathione Sepharose® 4B beads (GE Healthcare) were added to the supernatant and the mixture was rotated for 30 min at room temperature. The beads were then washed 3 times with ice-cold PBS and bound protein was eluted from the beads by the addition of reduced glutathione buffer (Amersham Biosciences). Eluted proteins were resolved by SDS-PAGE along with a standard curve of BSA. The gel was stained with Simply Blue SafeStain (Invitrogen) as per manufacturer's instructions. The protein was quantified using ChemiDoc XRS imaging system with Quantity 1 software (BioRad). Using this technology, mg amounts of soluble GST-E3L protein was obtainable.

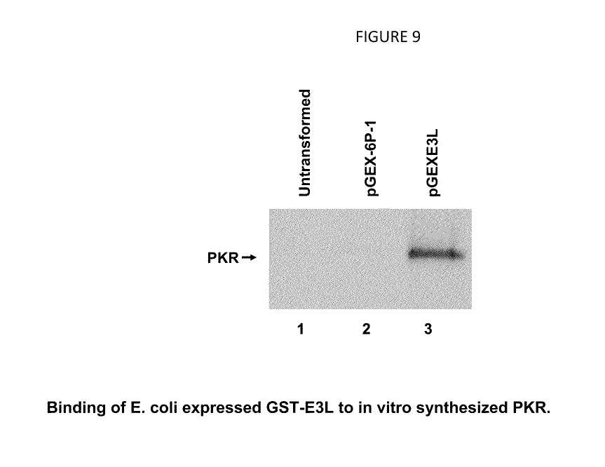

To assess function of the purified GST-E3L protein pulldown assays and EMSA assays were performed. E3L proteins can bind to the cellular protein PKR in vitro, in yeast (25) and in virus infected cells (our unpublished observations). GST-E3L protein, purified as described above, was incubated with 35S-PKR protein that had been synthesized in vitro. Complexes were pulled down with glutathione Sepharose and bound PKR was identified by on the radiography. As shown in FIG. 9, GST-E3L proteins purified from E. coli retain the ability to bind to PKR.

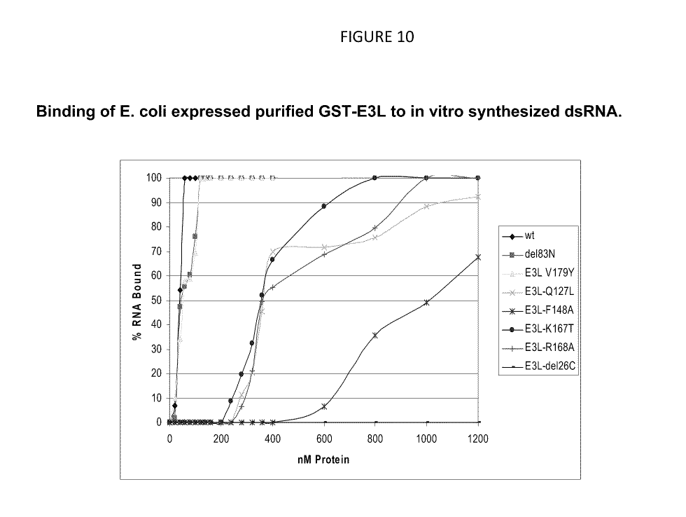

An EMSA assay was used to evaluate the ability of E. coli expressed GST-E3L protein to bind to dsRNA. Large amounts of dsRNA were synthesized using the RiboMAX Large Scale RNA Production System (Promega). Complimentary RNAs obtained with this method were annealed and treated with RNaseA to remove any ssRNA. Purified GST-E3L protein was incubated with the in vitro synthesized dsRNA and complexes were resolved by native PAGE. As can be seen in FIG. 10, purified GST-E3L protein retained the ability to bind to dsRNA.

These experiments demonstrate the feasibility of synthesis of pure, active GST-E3L protein from E. coli and the feasibility of loading this purified protein with in vitro synthesized dsRNA.

### LITERATURE CITED

1. Carrera, M. R., et al. 1995. Suppression of psychoactive effects of cocaine by active immunization. Nature 378:727-30.
2. Carrera, M. R., J. A. Ashley, P. Wirsching, G. F. Koob, & K. D. Janda. 2001. A second-generation vaccine protects against the psychoactive effects of cocaine. Proc Natl Acad Sci USA 98:1988-92.
3. Carroll, M. E., C. P. France, & R. A. Meisch. 1981. Intravenous self-administration of etonitazene, cocaine and phencyclidine in rats during food deprivation and satiation. J Pharmacol Exp Ther 217:241-7.
4. Diebold, S S 2009 Activation of dendritic cells by toll-like receptors and C-type lectins Handb Exp Pharm:3.
5. Freche, B., N. Reig, & F. G. van der Goot. 2007. The role of the inflammasome in cellular responses to toxins and bacterial effectors. Semin Immunopathol 29:249-60.
6. Gaucher, D., et al. 2008. Yellow fever vaccine induces integrated multilineage and polyfunctional immune responses. J Exp Med 205:3119-31.
7. Gerster, J. F., et al. 2005. Synthesis and structure-activity-relationships of 1H-imidazo[4,5-c]quinolines that induce interferon production. J Med Chem 48:3481-91.
8. Gupta, A. K., M. Browne, & R. Bluhm. 2002. Imiquimod: a review. J Cutan Med Surg 6:554-60.
9. Hrafnkelsdottir, K., J. Valgeirsson, & S. Gizurarson. 2005. Induction of protective and specific antibodies against cocaine by intranasal immunisation using a glyceride adjuvant. Biol Pharm Bull 28:1038-42.
10. Izumi, T., et al. 2003. 1H-Imidazo[4,5-c]quinoline derivatives as novel potent TNF-alpha suppressors: synthesis and structure-activity relationship of 1-, 2- and 4-substituted 1H-imidazo[4,5-c]quinolines or 1H-imidazo[4,5-c]pyridines. Bioorg Med Chem 11:2541-50.
11. Jasani, B., H. Navabi, & M. Adams. 2009. Ampligen: A potential toll-like 3 receptor adjuvant for immunotherapy of cancer. Vaccine In Press, Corrected Proof.
12. Johnston, D., B. Zaidi, & J. C. Bystryn. 2007. TLR7 imidazoquinoline ligand 3M-019 is a potent adjuvant for pure protein prototype vaccines. Cancer Immunol Immunother 56:1133-41.
13. Kantak, K. M. 2003 Anti-cocaine vaccines:antibody protection against relapse. ExpertOpinPharmac 4:213-8.
14. Khodair, A. I., M. M. A. Abbasi, E. I. Ibrahim, A. H. Soliman, & E. S. H. El Ashry. 1999. Synthesis of substituted quinolines and heterocyclo[x,y-c]quinolines by the nucleophilic substitution and rearrangements of 4-chloro-2-methyl-3-nitroquinolines. Heterocycl. Commun. 5:577.
15. Kosten, T., & S. M. Owens. 2005 Immunotherapy for the treatment of drug abuse. Pharmac Ther 108:76-85.
16. Langland, J. O., et al. 2006. Suppression of proinflammatory signal transduction and gene expression by the dual nucleic acid binding domains of the vaccinia virus E3L proteins. J Virol 80:10083-95.
17. Lemus, R., & M. H. Karol. 2008. Conjugation of haptens. Methods Mol Med 138:167-82.
18. Magalhaes, P. O., et al. 2007. Methods of endotoxin removal from biological preparations: a review. J Pharm Pharm Sci 10:388-404.
19. Martell, B. A., E. Mitchell, J. Poling, K. Gonsai, & T. R. Kosten. 2005. Vaccine pharmacotherapy for the treatment of cocaine dependence. Biol Psychiatry 58:158-64.
20. Meijler, M. M., et al. 2005. Fluorescent cocaine probes: a tool for the selection and engineering of therapeutic antibodies. J Am Chem Soc 127:2477-84.
21. Neisewander, J. L., et al. 2000. Fos protein expression and cocaine-seeking behavior in rats after exposure to a cocaine self-administration environment. J Neurosci 20:798-805.
22. Orson, F. M., et al. 2008. Substance abuse vaccines. Ann N Y Acad Sci 1141:257-69.
23. Parsons, L. H., et al. 1996. Neurochemical evidence that postsynaptic nucleus accumbens D3 receptor stimulation enhances cocaine reinforcement. J Neurochem 67:1078-89.
24. Phillips, M. A. 1928. The formation of 2-substituted benzimidazoles. J. Chem. Soc. 13:2393-2399.
25. Romano, P. R., et al. 1998 Inhibition of double-stranded RNA-dependent protein kinase PKR by vaccinia virus E3: role of complex formation and the E3 N-terminal domain. Mol Cell Biol 18:7304-16.
26. Sharp, F. A., et al. 2009. Uptake of particulate vaccine adjuvants by dendritic cells activates the NALP3 inflammasome. Proc Natl Acad Sci USA 106:870-5.
27. Tang, W., & S. Fang. 2008. Mono-acylation of symmetric diamines in the presence of water. Tetrahedron Letters 49:6003-6006.
28. Werling, D., & T. W. Jungi. 2003. TOLL-like receptors linking innate and adaptive immune response. Vet Immunol Immunopathol 91:1-12.
29. Wille-Reece, U., et al. 2005. HIV Gag protein conjugated to a Toll-like receptor 7/8 agonist improves the magnitude and quality of Th1 and CD8+ T cell responses in nonhuman primates. Proc Natl Acad Sci USA 102:15190-4.

1. A conjugate comprising at least one antigen or small molecule conjugated to at least one polypeptide capable of binding a double-stranded polynucleotide, wherein the conjugate is able to elicit an immune response against the antigen or small molecule.

2. The conjugate of claim 1, wherein the double-stranded polynucleotide comprises a double-stranded polyribonucleotide (dsRNA).

3. The conjugate of claim 1, wherein the at least one polypeptide capable of binding a double-stranded polynucleotide comprises a binding domain of a protein selected from the group consisting of: ADAR1, ZBP1, PKR-like kinase, and E3L.

4. The conjugate of claim 1 wherein the double stranded polyribonucleotide (dsRNA) comprises a first ligand.

5. The conjugate of claim 4 wherein the first ligand is biotin.

6. The conjugate of claim 4 further comprising a second ligand capable of binding to the first ligand.

7. The conjugate of claim 6 wherein the second ligand is a multimer, capable of binding more than one first ligand.

8. The conjugate of claim 7 wherein the second ligand comprises a tetrameric avidin.

9. The conjugate of claim 7 further comprising at least one adjuvant and/or at least one TLR agonist having a third ligand bound thereto, wherein the at least one adjuvant and/or at least one TLR agonist binds to the second ligand through interaction of the third ligand with the second ligand, and wherein the second ligand further binds to the dsRNA through interaction of the first and second ligand.

10. The conjugate of claim 9 wherein the first ligand comprises biotin and the second ligand comprises a tetrameric avidin, and wherein the third ligand comprises a biotin.

11. The conjugate of claim 11 wherein the polypeptide comprises E3L.

12. The conjugate of claim 9 wherein the at least one TLR agonist is selected from the group consisting of a TLR 4 agonist, a TLR9 agonist, a TLR 5 agonist, and a TLR 7/8 agonist.

13. The conjugate of claim 9 wherein the TLR agonist is selected from the group consisting of LPS, unmethylated CpG DNA, resiquimod and flagellin.

14. The conjugate of claim 1, wherein the at least one polypeptide comprises a Z-alpha domain or a variant thereof.

15. The conjugate of claim 1, wherein the at least one antigen is a disease-associated antigen.

16. A composition comprising the conjugate of claim 1 and a pharmaceutically acceptable carrier.

17. A DNA or RNA molecule encoding the conjugate of claim 1.

18. A host cell comprising the DNA or RNA molecule of claim 17.

19. A method for eliciting an immune response to an antigen in a subject, the method comprising administering to the subject the composition according to claim 16.

20. The conjugate of claim 1 wherein the small molecule comprises cocaine and wherein the polypeptide comprises E3L.

21. The conjugate of claim 13 wherein the small molecule comprises cocaine, and wherein the polypeptide comprises E3L and wherein the TLR agonist comprise unmethylated CPG DNA.

22. A kit comprising
a) a polypeptide capable of binding a double-stranded polynucleotide;
b) a dsRNA labeled with a first ligand; and
c) a second ligand that is able to bind to the first ligand.

23. The kit of claim 22 wherein the first ligand comprises biotin and the second ligand comprise a tetrameric avidin.

24. The kit of claim 22 further comprising at least one adjuvant and/or TLR agonist comprising a third ligand wherein the third ligand is able to bind the second ligand.

25. The kit of claim 24 wherein first ligand comprises biotin and the second ligand comprises tetrameric avidin and the third ligand comprises biotin.
